(19) United States
(12) Patent Application Publication
(10) Pub. No.: US 2012/0003219 A1
(43) Pub. Date: Jan. 5, 2012

(54) Compositions and Methods to Treat Bone Related Disorders

(75) Inventors: Chris LU, Lexington, MA (US); Shou-Ih HU, New Providence, NJ (US); Michaela KNEISSEL, Basel (CH); Christine HALLEUX, Dornach (CH)

(73) Assignee: NOVARTIS AG, Basel (CH)

(21) Appl. No.: 13/225,966

(22) Filed: Sep. 6, 2011

Related U.S. Application Data
(62) Division of application No. 12/525,631, filed on Aug. 3, 2009, now abandoned.
(60) Provisional application No. 60/887,956, filed on Feb. 2, 2007.

(51) Int. Cl. A61K 39/395; A61P 35/00; A61P 19/00; A61P 19/10; A61K 31/713; A61K 38/17

(52) U.S. Cl. 424/133.1; 514/44 A; 514/16.7; 514/16.9; 514/19.3; 424/172.1; 424/143.1

(57) ABSTRACT

The present invention relates to the use of modulators of the sclerostin:sclerostin-binding-partner interaction for the treatment, amelioration, and diagnosis of sclerostin-related disorders, e.g., osteoporosis and sclerosteosis, and sclerostin-related disorders, e.g., cancers and cardiovascular disorders. The invention also relates to the use of sclerostin-binding-partner mimetics for the treatment, amelioration, and diagnosis of sclerostin-related disorders. Assays for the identification of modulators of the sclerostin:sclerostin-binding-partner interaction, as well as the resulting signaling, are also provided.

### PRIORITY INFORMATION

This U.S. divisional application claims priority U.S. PCT application Ser. No. 12/525,631, 371(c) date: 3 Aug. 2009 which claims priority to U.S. PCT Application Serial No. PCT/EP08/051,128, filed: 30 Jan. 2008 and U.S. Provisional Application Ser. No. 60/887,956, filed: 2 Feb. 2007, the contents of which are incorporated herein by reference in their entirety.

### BACKGROUND OF THE INVENTION

Osteoporosis (OP) is a systemic skeletal disorder characterized by low bone mass and micro-architectural deterioration of bone tissue, with a consequent increase in bone fragility and susceptibility to fracture. It is estimated that a 50 year old woman has a 50% chance of having an osteoporotic fracture over her postmenopausal lifetime. The osteoporotic syndrome is multifaceted, encompassing primary disorders such as postmenopausal or age-related OP, and secondary conditions that accompany disease states or medications. Low bone mineral density (BMD) is the most important risk factor for OP. It results from impaired peak bone acquisition in adolescence or bone loss during aging.

Bone loss can occur as the result of accelerated bone resorption or defective bone formation, two processes which are normally coupled in adulthood. Rapid bone loss as a result of estrogen deficiency and accelerated bone resorption is the most frequent cause of OP, but only about one fourth of all early postmenopausal women have high bone turnover osteoporosis. Thus osteoporosis is largely caused by genetic factors. Several linkage analyses have identified genes contributing to enhanced bone turnover or low bone mass, but it is clear that no single gene defect accounts for, e.g., postmenopausal OP, the most frequent form of the disease (Ralston S H. (2003) Curr. Opin. Pharmacol. 3(3):286).

Current prevalence of osteoporosis is 50.9 million patients in major markets, expected to rise to 55.8 million by 2010 and 61.5 million by 2015. There is a steady increase due to aging of the population.

Most current therapies for the treatment of OP are based on inhibiting bone resorption to prevent further bone loss. The reason for this is that the bone resorbing (degrading) cell—the osteoclast—is a highly specialized cell specific to bone, and key mechanisms in its recruitment and activation have been identified. Osteoclasts are cells of hemopoietic origin and develop from stem cells in the bone marrow; mature functional osteoclasts are multinuclear cells and are localized on mineralized bone surfaces that these specialized cells can resorb.

New bone matrix is formed by osteoblasts, which stem from mesenchymal lineage. These bone forming cells have three putative final fates: they undergo apoptosis (cell death), become lining cells (flat cells on the mineralized bone surface), or are entrapped into the bone matrix. The entrapped terminally-differentiated cells are called osteocytes, which are the most abundant bone cells. They are embedded at regular intervals within the mineralized bone matrix and are interconnected with each other through long cellular processes termed dendrites, which are coupled to neighboring osteocytes and bone cells on the surface via gap junctions. They are thought to be key regulators of bone modeling and remodeling, though the underlying molecular mechanisms are not fully understood.

Although osteoporosis has been defined as an increase in the risk of fracture due to decreased bone mass, none of the presently available treatments for skeletal disorders can substantially increase the bone density of adults. Current osteoporotic therapeutic principles instead are anti-resorptive, and are capable of reducing the risk for new vertebral fractures by about 35 to 60% (Haeuselmann H J, Rizzoli R (2003) Osteoporos. Int.; 14:2; Chesnut C H, III, et al (2004). J Bone Min Res; 19 (8)). Hip fracture risk is only reduced by one anti-resorptive therapeutic principle, resulting in about a 50% reduction in fracture risk. A longstanding unmet need in the field is represented by drugs capable of increasing bone density in adults, particularly in the bones of the wrist, spinal column and hip that are at risk in osteopenia and osteoporosis. Accordingly, because many OP patients have already lost a substantial amount of bone at the time of diagnosis, there is a need for developing agents that increase bone mass by stimulating new bone formation, and which should be able to reduce fracture risk by more than 50%.

Conversely, there are a variety of bone disorders associated with bone overgrowth and aberrantly high bone mineral density (BMD), in which bone formation and deposition exceed resorption, potentially resulting in pathologically increased bone mass and strength. For example, sclerosteosis is a recessive disorder that exhibits increased bone mass even in heterozygous carriers. Likewise, subjects with Simpson-Golabi-Behmel syndrome (SGBS) typically have a broad, stocky appearance, and are characterized by enlarged facial bones (e.g., resulting in protruding jaw and enlarged nasal bridge). Similarly, Van Buchem Disease is an autosomal recessive disease characterized by skeletal overgrowth. This disease is characterized by a symmetrically increased thickness of bones, most frequently found as an enlarged jawbone, but also an enlargement of the skull, ribs, diaphysis of long bones, as well as tubular bones of hands and feet, resulting in increased cortical bone density. The clinical consequences of increased thickness of the skull include facial nerve palsy causing hearing loss, visual problems, neurological pain, and, very rarely, blindness as a consequence of optic atrophy.

The present invention aims to provides compositions, methods of treatment, and methods of diagnosis of disorders related to abnormal bone mineral density (BMD).

### SUMMARY OF THE INVENTION

The present invention provides methods of treating, ameliorating the symptoms of, and protecting against an aberrant bone mineral density disorder and/or a sclerostin-related disorder comprising administering a composition comprising a modulator of the sclerostin-binding-partner interaction, for example a modulator of the sclerostin:sclerostin-binding partner interaction. Said modulator can include an antibody, an Antibody-like Scaffold, small molecule, chimeric or fusion protein, peptide, mimetic, or inhibitory nucleotide (e.g., RNAi) directed against (i) sclerostin; (ii) a sclerostin-binding-partner; (iii) a novel site (e.g., a newly created epitopic determinant) created by the sclerostin:sclerostin-binding-partner interaction, or (iv) a protein complex comprising any of the same. Said compositions include “pharmaceutical compositions,” as defined herein. “Sclerostin-related disorder,” “aberrant bone mineral density disorder” and “sclerostin-binding-partner” are terms further defined herein.

By way of non-limiting example, the present invention provides methods of treating a sclerostin-related disorder (e.g., osteoporosis or sclerosteosis) and/or an aberrant bone mineral density disorder by administering a modulator of the sclerostin:ALPL, sclerostin:Frem2 or sclerostin:LRP4 interaction.

The present invention further provides methods for identifying an agent capable of modulating the sclerostin:sclerostin-binding-partner interaction, which method comprises measuring the alteration of sclerostin interaction with a sclerostin-binding-partner occasioned by said agent. Preferably said method comprises the steps of: a) contacting sclerostin with a sclerostin-binding-partner in the presence and absence of a test agent under conditions permitting the interaction of the sclerostin-binding-partner with sclerostin; and b) measuring interaction of the sclerostin-binding-partner with sclerostin in both the presence and absence of said test agent wherein (i) a decrease in sclerostin:sclerostin-binding-partner interaction in the presence of the test agent, relative to the interaction in the absence of the test agent, identifies the test agent as an antagonist of the sclerostin:sclerostin-binding-partner interaction, and wherein (ii) an increase in the interaction in the presence of the test agent, relative to the interaction in the absence of the test agent, identifies the test agent as an agonist of the sclerostin:sclerostin-binding-partner interaction.

By way of non-limiting example, the present invention provides methods for identifying an agent capable of modulating the sclerostin:LRP4 interaction, which method comprises measuring the alteration of sclerostin binding to LRP4 occasioned by said agent. In one embodiment, the test agent binds directly or indirectly to LRP4 and thereby modulates the sclerostin:LRP4 interaction. In one embodiment, the test agent is small molecule. In another embodiment, the test agent is an inhibitory nucleotide. In still another embodiment, the test agent is a fusion protein comprising the LRP4 protein.

Alterations in sclerostin:sclerostin-binding-partner interaction, sclerostin or sclerostin-binding-partner protein activity, and/or sclerostin pathway activity may be measured by PCR, Taqman PCR, phage display systems, gel electrophoresis, yeast-two hybrid assay, reporter gene assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine. The change in sclerostin, sclerostin-binding-partner protein activity, and/or sclerostin pathway activity may be detected by detecting a change in the interaction between sclerostin:sclerostin-binding-partner, by detecting a change in the expression or protein level of sclerostin or sclerostin-binding-partner, or by detecting a change in the expression or protein level of one or more of the proteins in the sclerostin pathway, preferably by detecting a change in the Wnt signaling pathway. Cells in which the above-described may be detected can be of bone, mesenchymal, kidney (e.g., HEK), or hematopoietic origin, may be cultured cells, or may be obtained from or may be within a transgenic organism. Such transgenic organisms include, but are not limited to a mouse, rat, rabbit, sheep, cow or primate.

The present invention also provides methods for identifying an agent capable of modulating the sclerostin:sclerostin-binding-partner interaction, which method comprises measuring the signaling response induced by the sclerostin:sclerostin-binding-partner interaction in the presence of said agent, and comparing it with the signaling response induced by the sclerostin:sclerostin-binding-partner interaction in the absence of said agent. Preferably, said method comprises the steps of: a) contacting sclerostin with a sclerostin-binding-partner in the presence and absence of a test agent under conditions permitting the interaction of the sclerostin-binding-partner with sclerostin; and b) measuring a signaling or enzymatic response induced by the sclerostin:sclerostin-binding-partner interaction, wherein a change in response in the presence of the test agent of at least 10%, 20% or 30% compared with the response in the absence of the test agent indicates the test agent is capable of modulating the sclerostin:sclerostin-binding-partner interaction. Preferably, the signaling response measured at step b) is the Wnt signaling response.

An increase in signaling response in the presence of the test agent of at least 10%, 20% or 30% compared with the response in the absence of the test agent identifies the test agent as an agonist of the sclerostin:sclerostin-binding-partner interaction. A decrease in signaling response in the presence of the test agent of at least 10%, 20% or 30% compared with the response in the absence of the test agent identifies the test agent as an antagonist of the sclerostin:sclerostin-binding-partner interaction.

By way of non-limiting example, the present invention provides methods for identifying an agent capable of modulating the sclerostin:LRP4 interaction, which method comprises measuring the signaling response induced by the sclerostin:LRP4 interaction in the presence of said agent, and comparing it with the signaling response induced by the sclerostin:LRP4 interaction in the absence of said agent. In one embodiment, the test agent binds directly or indirectly to LRP4 and thereby modulates the sclerostin:LRP4 interaction. In one embodiment, the test agent is small molecule. In another embodiment, the test agent is an inhibitory nucleotide.

Also, the present invention provides a composition comprising an agent capable of modulating the sclerostin:sclerostin-binding-partner interaction identified according to a method as described above. Said compositions include “pharmaceutical compositions,” as defined herein.

Preferred agent capable of modulating the sclerostin-binding partner are antibodies or Antibody-like Scaffolds that bind specifically to said sclerostin-binding partner or a functional protein comprising an antigen-binding portion of said antibody or said antibody-like scaffold.

The present invention also provides methods for diagnosing a sclerostin-related disorder and/or an aberrant bone mineral density disorder, or a predisposition to a sclerostin-related disorder and/or an aberrant bone mineral density disorder in a subject comprising the steps of (a) measuring the sclerostin:sclerostin-binding-partner interaction in said subject, and (b) comparing the interaction in step (a) with the sclerostin:sclerostin-binding-partner interaction of a healthy individual, a difference indicating a sclerostin-related disorder or predisposition thereto in said subject.

By way of non-limiting example, the present invention provides methods for diagnosing osteoporosis or sclerosteosis, or a predisposition thereto, in a subject by (a) measuring the sclerostin:LRP4 binding in said subject, and (b) comparing the binding in step (a) with the sclerostin:LRP4 binding of a healthy individual (i.e., one without an affliction or predisposition to sclerosteosis), a difference indicating osteoporosis or sclerosteosis or predisposition thereto in said subject.

The present invention provides a method for identifying a sclerostin-binding-partner mimetic, which mimetic has the same, similar or improved functional effect as sclerostin-binding-partner interaction with sclerostin, wherein the method comprises measuring the interaction with sclerostin by a candidate mimetic. Preferably, said method comprises: (a) contacting sclerostin with a candidate mimetic under conditions permitting the interaction of the mimetic with sclerostin; and (b) measuring interaction of the mimetic with sclerostin, wherein an interaction at least 10%, 20% or 30% of that observed for the various sclerostin:sclerostin-binding-partner interactions described herein distinguishes the candidate mimetic as a sclerostin-binding-partner mimetic of the invention.

Furthermore, the present invention provides a method for identifying a sclerostin-binding-partner mimetic, which mimetic has the same, similar or improved functional effect as sclerostin-binding-partner interaction with sclerostin, wherein the method comprises measuring the signaling response induced by the sclerostin-mimetic interaction and comparing it with the signaling response induced by sclerostin:sclerostin-binding-partner interactions described herein. Preferably, said method comprises: (a) contacting sclerostin with a candidate mimetic under conditions permitting the interaction of the mimetic with sclerostin; and (b) measuring a signaling response induced by the sclerostin-mimetic interaction, wherein a signaling response that is at least 10%, 20% or 30% of that observed for the various sclerostin:sclerostin-binding-partner interactions described herein distinguishes the candidate mimetic as a sclerostin-binding-partner mimetic of the invention.

By way of non-limiting example, the present invention provides methods for identifying LRP4 mimetics that have the same, similar or improved functional effects as those of the interaction between LRP4 and sclerostin under normal physiological conditions. Said mimetics can be identified by employing the methods described herein.

The present invention further provides siRNA capable of modulating protein expression, for example, decreasing protein expression in a mammalian cell of a sclerostin-binding partner.

Also, the present invention provides a composition comprising a sclerostin-binding-partner mimetic identified according to a method as described above. Said compositions include “pharmaceutical compositions,” as defined herein.

The present invention provides methods of treating, ameliorating the symptoms of, and protecting against a sclerostin-related disorder and/or an aberrant bone mineral density disorder comprising administering a composition comprising a sclerostin-binding-partner mimetic, which mimetic has the same, similar or improved functional effect as the sclerostin-binding-partner interaction with sclerostin described herein. Said mimetics can easily be identified by using the methods described above (and in further detail herein). In certain embodiments, said mimetic can be an antibody or Antibody-like Scaffold or fragment thereof directed against said sclerostin-binding-partner (e.g., anti-LRP4 antibody or fragment).

The present invention provides additionally a method for diagnosing a disorder or predisposition to a sclerostin-related disorder and/or an aberrant bone mineral density disorder in a subject comprising the steps of: (a) obtaining the nucleotide sequence of a sclerostin-binding-partner gene in said subject, and (b) comparing it to that of a healthy subject, where a mutation in the respective sclerostin-binding-partner gene indicates a sclerostin-related disorder and/or an aberrant bone mineral density disorder or a predisposition thereto.

The present invention also provides methods of modulating the interaction between sclerostin and a sclerostin-binding-partner. By way of non-limiting example, the present invention includes methods of modulating the interaction between sclerostin and a sclerostin-binding-partner in order to modulate sclerostin pathway activity, or to modulate sclerostin or sclerostin-binding-partner protein levels.

The present invention is not limited to the native sequence of sclerostin or any of the sclerostin-binding-partners described in detail herein. Furthermore, the methods and compositions of the present invention encompass derivatives and splice variants of sclerostin or any of the sclerostin-binding-partners described in detail herein. Even where portions or fragments are employed, these portions or fragments may have altered amino acid sequences.

The present invention further provides a soluble polypeptide comprising a fragment of sclerostin-binding partner, wherein said soluble polypeptide binds specifically to a sclerostin-binding partner, for example LRP4, or sclerostin. In one embodiment, said soluble polypeptide consists of the extracellular portion of LRP4, preferably the polypeptide consisting of SEQ ID NO 3 (LRP4 aa 21-1763).

The present invention also provides an antibody or Antibody-like Scaffold or functional protein comprising an antigen-binding portion of said antibody or said Antibody-like Scaffold, wherein said antibody or Antibody-like Scaffold or functional protein binds specifically to a sclerostin-binding partner. In one embodiment, said antibody or Antibody-like Scaffold or functional protein inhibits binding of sclerostin to said sclerostin-binding partner. In another embodiment, said antibody or Antibody-like Scaffold or functional protein modulates the Wnt signaling pathway, as measured in a cell-based assay. In one related embodiment, said antibody or Antibody-like Scaffold or functional protein binds specifically to LRP4 or ALPL.

### BRIEF DESCRIPTION OF THE FIGURES

FIG. 1 shows anti-LRP4 siRNAs, and their ability to knockdown LRP4 mRNA.

FIG. 2 shows that the LRP4 mRNA knockdown reduces the ability of sclerostin to inhibit supertopflash (STF) activity in HEK293 cells.

FIG. 3 shows a sclerostin dose response study after LRP4 mRNA knockdown.

FIG. 4a shows the effect of LRP4 overexpression on Wnt1-induced supertopflash (STF)-Luc in Hek293 cells; FIG. 4b shows the effect of LRP4 overexpression on the action of sclerostin and Dkk1 on Wnt1-induced supertopflash (STF)-Luc in Hek293 cells; FIG. 4c shows the effect of LRP4 and LRP5 overexpression on the action of sclerostin and Dkk1 on Wnt1-induced supertopflash (STF)-Luc in Hek293 cells; FIG. 4d shows the effect of LRP4 and LRP6 overexpression on the action of sclerostin and Dkk1 on Wnt1-induced supertopflash (STF)-Luc in Hek293 cells.

FIG. 5a shows the effect of LRP4 overexpression on Wnt1-induced supertopflash (STF)-Luc in C28a2 cells; FIG. 5b shows the effect of LRP4 overexpression on the action of sclerostin and Dkk1 on Wnt1-induced supertopflash (STF)-Luc in C28a2 cells

FIG. 6 shows the effect of sclerostin on GSK3beta inhibitor-induced alkaline phosphatase activity in MC3T3 cells

FIG. 7 shows the effect of sclerostin on alkaline phosphatase activity in a cell-free based assay

### DETAILED DESCRIPTION OF THE INVENTION

In the present description, the term “treatment” includes both prophylactic or preventive treatment as well as curative or disease suppressive treatment, including treatment of patients predisposed to illness (e.g., to a sclerostin-related disorders and/or aberrant bone mineral density disorders) as well as ill patients. This term further includes the treatment for the delay of progression of the disease.

As used herein, “sclerostin-binding-partner” includes, but is not limited to the following proteins: Versican (CSPG2), FREM2, Fibrillin 2 (FBN2), C6orf93, Syndecan-4 (Sdc4), Agrin (AGRN), Serpine-2 (PN-1), LRP2, LRP4, LRP6, SLIT2, tenascin C, TRIM26, TRIM41, glypican1, alkaline phosphatase (ALPL) and IL-17 receptor. In one embodiment, LRP4 and ALPL refers to corresponding human LRP4 and ALPL having SEQ ID NO:1 and 2 respectively.

“Sclerostin:sclerostin-binding-partner interaction” means a direct or indirect interaction between sclerostin and a sclerostin-binding-partner as defined herein. Non-limiting examples of interactions include direct physical binding and indirect steric inhibition.

As used herein, “a sclerostin-related disorder” includes disorders in which bone mineral density (BMD) is abnormally and/or pathologically high relative to healthy subjects, and disorders in which bone mineral density (BMD) is abnormally and/or pathologically low relative to healthy subjects. Disorders characterized by high BMD include but are not limited to sclerosteosis, Van Buchem disease, bone overgrowth disorders, and Simpson-Golabi-Behmel syndrome (SGBS). Disorders characterized by low BMD and/or bone fragility include but are not limited to primary and secondary osteoporosis, osteopenia, osteomalacia, osteogenesis imperfecta (OI), avascular necrosis (osteonecrosis), fractures and implant healing (dental implants and hip implants), bone loss due to other disorders (e.g., associated with HIV infection, cancers, or arthritis). Other “sclerostin-related disorders” include but are not limited to rheumatoid arthritis, osteoarthritis, arthritis, and the formation and/or presence of osteolytic lesions.

As used herein, “a sclerostin-related disorder” includes conditions mediated by sclerostin or associated with or characterized by aberrant sclerostin levels. These include cancers and osteoporotic conditions (e.g., osteoporosis or osteopenia), some of which overlap with “sclerostin-related disorders” as defined herein. Sclerostin-related cancers can include myeloma (e.g., multiple myeloma with osteolytic lesions), breast cancer, colon cancer, melanoma, hepatocellular cancer, epithelial cancer, esophageal cancer, brain cancer, lung cancer, prostate cancer, or pancreatic cancer, as well as any metastases thereof.

A “sclerostin-related disorder” can also include renal and cardiovascular conditions, due at least to sclerostin's expression in the kidney and cardiovasculature. Said disorders include but are not limited to such renal disorders as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

Said disorders also include but are not limited to such cardiovascular disorders as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

“Cure” as used herein means to lead to the remission of the disorder, e.g., the sclerostin-related disorder and/or aberrant bone mineral density disorder, or of ongoing episodes thereof, through treatment.

The terms “prophylaxis” or “prevention” means impeding the onset or recurrence of a sclerostin-related disorder and/or an aberrant bone mineral density disorder, e.g., osteoporosis, sclerosteosis, or cancer.

As used herein, “modulate” indicates the ability to control or influence directly or indirectly, and by way of non-limiting examples, can alternatively mean inhibit or stimulate, agonize or antagonize, hinder or promote, and strengthen or weaken.

As used herein a “small organic molecule,” or “small molecule,” is an organic compound (or organic compound complexed with an inorganic compound (e.g., metal) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as luciferase.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may be used to prepare pharmaceutical and diagnostic compositions, or that may be compounds, nucleic acids (including inhibitory nucleic acids such as shRNA, RNAi, etc.), antibodies, Antibody-like Scaffolds, small molecules, polypeptides, fragments, isoforms, variants, or other materials that may be used independently for such purposes, all in accordance with the present invention.

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

“Inhibitors,” or “antagonists” refer to inhibitory molecules, including those identified using the sclerostin:sclerostin-binding-partner screening methods described herein, of sclerostin and/or sclerostin-binding-partner activity, or of the activity of related proteins or pathways (e.g., BMP, Wnt, etc.). Inhibitors and antagonists may be agents that decrease, block, or prevent, signaling via a pathway and/or which prevent the formation of protein interactions and complexes.

“Mimetic,” according to the present invention, includes, but is not limited to a polypeptide, a peptide, a lipid, a carbohydrate, a nucleotide, a small organic molecule, and an antibody or antigen-binding fragment thereof. Mimetics can be used to mirror (or enhance) the activity of a protein, peptide, or polypeptide of interest (e.g., sclerostin or a sclerostin-binding-partner), in order to, for example, replicate, agonize, or potentiate the effects of the sclerostin:sclerostin-binding-partner interaction.

Candidate mimetics can be natural or synthetic compounds, including, for example, synthetic small molecules, compounds contained in extracts of animal, plant, bacterial or fungal cells, as well as conditioned medium from such cells. Mimetic compounds can be determined using the methods described below. Mimetics can be generated based on a knowledge of the critical residues of a subject protein, peptide, polypeptide which can mimic normal polypeptide function. A mimetic can have the same, similar or improved functional effects as the polypeptide, peptide, or protein after which it is designed.

The term “double-stranded RNA” or “dsRNA,” as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where separate RNA molecules, such siRNA are often referred to in the literature as siRNA (“short interfering RNA”). Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop,” “short hairpin RNA,” or “shRNA.” Where the two strands are connected covalently by means other than an uninterrupted chain of nucleotides between the 3′-end of one strand and the 5′end of the respective other strand forming the duplex structure, the connecting structure is referred to as a “linker”. The RNA strands may have the same or a different number of nucleotides. The maximum number of base pairs is the number of nucleotides in the shortest strand of the siRNA minus any overhangs that are present in the duplex. In addition to the duplex structure, a siRNA may comprise one or more nucleotide overhangs. In addition, as used in this specification, “siRNA” may include chemical modifications to ribonucleotides, including substantial modifications at multiple nucleotides and including all types of modifications disclosed herein or known in the art. Any such modifications, as used in an siRNA type molecule, are encompassed by “siRNA” for the purposes of this specification and claims.

As used herein, a “nucleotide overhang” refers to the unpaired nucleotide or nucleotides that protrude from the duplex structure of a siRNA when a 3′-end of one strand of the siRNA extends beyond the 5′-end of the other strand, or vice versa. “Blunt” or “blunt end” means that there are no unpaired nucleotides at that end of the siRNA, i.e., no nucleotide overhang. A “blunt ended” siRNA is a siRNA that is double-stranded over its entire length, i.e., no nucleotide overhang at either end of the molecule. For clarity, chemical caps or non-nucleotide chemical moieties conjugated to the 3′ end or 5′ end of an siRNA are not considered in determining whether an siRNA has an overhang or is blunt ended.

The term “antisense strand” refers to the strand of a siRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a siRNA that includes a region that is substantially complementary to a region of the antisense strand.

“Introducing into a cell”, when referring to a siRNA, means facilitating uptake or absorption into the cell, as is understood by those skilled in the art. Absorption or uptake of siRNA can occur through unaided diffusive or active cellular processes, or by auxiliary agents or devices. The meaning of this term is not limited to cells in vitro; a siRNA may also be “introduced into a cell”, wherein the cell is part of a living organism. In such instance, introduction into the cell will include the delivery to the organism. For example, for in vivo delivery, siRNA can be injected into a tissue site or administered systemically. In vitro introduction into a cell includes methods known in the art such as electroporation and lipofection.

The terms “silence” and “inhibit the expression of,” in as far as they refer to the SOST gene (i.e., the gene which encodes sclerostin), the genes encoding sclerostin-binding-partners (e.g., the genes encoding Versican (CSPG2), FREM2, Fibrillin 2 (FBN2), C6orf93, Syndecan-4 (Sdc4), Agrin (AGRN), Serpine-2 (PN-1), LRP2, LRP4, LRP6, SLIT2, tenascin C, TRIM26, TRIM41, glypican1, alkaline phosphatase (ALPL) and IL-17 receptor), or any genes involved in the sclerostin, BMP, or Wnt signaling pathways, herein refer to the at least partial suppression of the expression of said genes, as manifested by a reduction of the amount of mRNA transcribed from said genes which may be isolated from a first cell or group of cells in which said genes are transcribed and which has or have been treated such that the expression of said genes are inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the SOST gene, the genes encoding sclerostin-binding-partners, or any genes involved in the sclerostin, BMP, or Wnt signaling pathways transcription, e.g. the amount of protein encoded by said genes which is secreted by a cell, or the number of cells displaying a certain phenotype. In principle, silencing of the SOST gene, the genes encoding sclerostin-binding-partners, or any genes involved in the sclerostin, BMP, or Wnt signaling pathways may be determined in any cell expressing the target, either constitutively or by genomic engineering, and by any appropriate assay. However, when a reference is needed in order to determine whether a given siRNA inhibits the expression of the SOST gene, the genes encoding sclerostin-binding-partners, or any genes involved in the sclerostin, BMP, or Wnt signaling pathways by a certain degree and therefore is encompassed by the instant invention, the assay provided in the Examples below shall serve as such reference.

For example, in certain instances, expression of the SOST gene, the genes encoding sclerostin-binding-partners, or any genes involved in the sclerostin, BMP, or Wnt signaling pathways is suppressed by at least about 20%, 25%, 35%, or 50% by administration of the double-stranded oligonucleotide of the invention. In some embodiment, said genes are suppressed by at least about 60%, 70%, or 80% by administration of the double-stranded oligonucleotide of the invention. In some embodiments, said genes are suppressed by at least about 85%, 90%, or 95% by administration of the double-stranded oligonucleotide of the invention.

The term “binding” refers to the physical association of a component (e.g., sclerostin) with another component (e.g., sclerostin-binding-partner). A measurement of binding can lead to a value such as a dissociation constant, an association constant, on-rate or off-rate.

As used herein, the term “conditions permitting the binding.” refers to conditions of, for example, temperature, salt concentration, pH and protein concentration under which binding will arise. Exact binding conditions will vary depending upon the nature of the assay, for example, whether the assay uses pure proteins or only partially purified proteins. Temperatures for binding can vary from 15° C. to 37° C., but will preferably be between room temperature and about 30° C. The concentration of sclerostin in a binding reaction will also vary, but will preferably be about 10 μM to 10 nM (e.g., in a reaction using radiolabeled components).

As the term is used herein, binding is “specific” if it occurs with a Kd of 1 mM or less, generally in the range of 100 nM to 10 pM. For example, binding is specific if the Kd is 100 nM, 50 nM, 10 nM, 1 nM, 950 pM, 900 pM, 850 pM, 800 pM, 750 pM, 700 pM, 650 pM, 600 pM, 550 pM, 500 pM, 450 pM, 350 pM, 300 pM, 250 pM, 200 pM, 150 pM, 100 pM, 75 pM, 50 pM, 25 pM, 10 pM or less.

The present invention relates to the discovery of protein binders to sclerostin (referred to herein as “sclerostin-binding-partner(s)”). Sclerostin is capable of inhibiting bone deposition when bound to (or otherwise interacting with) said sclerostin-binding-partners, and in the absence of said binding or interaction, the inhibition of bone deposition partially recedes or increases depending whether the binding partners acts as a positive mediator or inhibitor of sclerostin action. Sclerostin is known in the art to modulate other proteins and signaling pathways. By way of example, studies have shown that sclerostin is capable of functioning as a context-dependent antagonist of Bone Morphogenic Protein (BMP) signaling. Furthermore, sclerostin can also function as an inhibitor of the Wingless/INT (Wnt) signaling pathway, possibly by binding to LRP5 and LRP6 Wnt co-receptors. Sclerostin's ability to modulate adult bone formation may occur via Wnt signaling inhibition, a theory supported by the high phenotypic overlap in human bone overgrowth disorders related to loss-of-function mutations in sclerostin and gain-of-function mutations in LRP5.

Because sclerostin is known in the art to modulate other proteins and signaling pathways, modulation of the sclerostin:sclerostin-binding-partner interaction likewise can influence and otherwise exert effects on other proteins and signaling pathways (e.g., Wnt and BMP). Modulation of the sclerostin:sclerostin-binding-partner interaction (e.g., by the methods and compositions of the present invention) can therefore result in altered sclerostin levels or increased or decreased bone density, and can be leveraged for the treatment of sclerostin-related disorders, or aberrant bone mineral density disorders, respectively. This is demonstrated in the Examples section herein.

While other factors have been implicated in bone deposition via the Wnt and BMP signaling pathways in past studies, the discoveries detailed herein (and embodied in the present invention) are critical in that they reveal modulation of these pathways by the sclerostin:sclerostin-binding-partner interaction. In other words, the sclerostin:sclerostin-binding-partner interaction is critical in order to either activate or inhibit sclerostin, whereby it is capable of achieving its biological activities (e.g., inhibiting bone deposition).

By way of example, fibrillin 2 is an important binding partner of sclerostin, or is part of a multi-complex consisting of at least sclerostin, as described herein. This interaction can induce a refolding of sclerostin into a more stable conformation, and/or can initiate the functional interaction between fibrillin-2 and BMP signaling, thereby providing a link between sclerostin and BMP signaling. Likewise, the interaction between fibrillin 2 and sclerostin can initiate the functional interaction between fibrillin-2 and Wnt signaling, thereby providing a link between sclerostin and Wnt signaling. ¹⁵N-sclerostin preparations reveal only 25% structuring by NMR, presumably due to sclerostin folding upon binding ligand(s) such as fibrillin 2.

These findings engender the methods and compositions of the present invention, which can, among other things, treat, prevent, and diagnose sclerostin-related disorders and/or aberrant bone mineral density disorders. For example, disorders characterized by aberrantly low bone mineral density (e.g., osteoporosis) may be prevented, treated, or ameliorated by modulating (e.g., disrupting) the sclerostin:sclerostin-binding-partner interaction.

Disrupting the sclerostin:fibrillin 2 interaction, for instance (e.g., through the use of an anti-fibrillin 2 antibody or inhibitory nucleotide) can be used to prevent, treat, or ameliorate osteoporosis. Alternatively, disorders characterized by aberrantly high bone mineral density (e.g., osteoporosis) may be prevented, treated, or ameliorated by modulating (e.g., enhancing or agonizing) the sclerostin:sclerostin-binding-partner interaction, or by administering a fibrillin 2 mimetic which has the same or similar functional effect as fibrillin 2 binding to sclerostin. Agonizing the sclerostin:fibrillin 2 interaction, for instance (e.g., through the provision of a fibrillin 2 mimetic), in such a way as to enhance sclerostin action, can be used to prevent, treat, or ameliorate sclerosteosis.

Furthermore, in many cases, the interaction between sclerostin and a sclerostin-binding-partner can initiate the functional interaction between the sclerostin-binding-partner and BMP signaling, thereby forming a link between sclerostin and BMP signaling. Likewise, the sclerostin:sclerostin-binding-partner interaction can initiate the functional interaction between the sclerostin-binding-partner and Wnt signaling, thereby providing the nexus between sclerostin and Wnt signaling.

For example, the interaction between sclerostin and agrin can initiate the functional interaction between agrin and BMP signaling, thereby forming a link between sclerostin and BMP signaling. Likewise, the sclerostin-agrin interaction can initiate the functional interaction between agrin and Wnt signaling, thereby providing the nexus between sclerostin and Wnt signaling.

Sclerostin/SOST

Sclerostin, a protein encoded by the SOST gene, is a potent negative regulator of bone formation secreted by osteocytes (Swiss-Prot accession no. Q9BQB4). Due to sclerostin's similarity in its cysteine-knot structure with the DAN family of TGF-β antagonists, sclerostin was originally hypothesized to be solely a bone morphogenic protein (BMP) antagonist (Brunkow et al (2001) Am J Hum Genet, 68:577-589); however, its ability to interact directly with BMPs in vivo has remained speculative.

Loss of sclerostin or SOST expression results in uncontrolled bone formation, e.g., as is the case with sclerosteosis (Brunkow et al. (2001) Am J Hum Genet.; 68(3):577). Patients afflicted with sclerosteosis endure life-long bone overgrowth resulting in increased bone mass and strength. Heterozygous carriers for this recessive disorder also display increased bone mass (Gardner et al. 2005 J Clin Endocrinol Metab. 90(12):6392). This phenotype can recapitulate in SOST deficient mice and its overexpression results in osteopenia (Loots et al. 2005 (Genome Res. 2005 15(7):928). Furthermore, Van Buchem disease—a phenotypic copy of sclerosteosis—has been found to be caused by SOST misregulation due to the genomic deletion of a long-range bone enhancer (Loots et al. 2005 (Genome Res. 2005 15(7):928).). Finally, studies show that SOST is down-regulated by parathyroid hormone—the only clinically validated bone forming principle—through the bone enhancer during bone formation (Keller, Kneissel 2005, Bone. 2005 37(2):148). Hence inhibition of sclerostin action should result in an ideal therapy for osteoporosis.

Although it remains unclear how exactly sclerostin exerts its action as a negative bone formation regulator, studies show that sclerostin inhibits bone morphogenetic protein (BMP) and Wingless/INT (WNT) signaling, both critical to bone formation. Sclerostin is capable of functioning as a context-dependent antagonist of BMP signaling. Furthermore, sclerostin can also function as an inhibitor of the Wnt signaling pathway, possibly by binding to Lrp5 and Lrp6 (Semenov M V, He X. J Biol. Chem. 2006; 281(50):3827) Wnt co-receptors in the presence of yet unidentified co-factor[s]. The hypothesis that sclerostin might impact adult bone formation by Wnt signaling inhibition is supported by the high phenotypic overlap in human bone overgrowth disorders related to loss-of-function mutations in sclerostin and gain-of-function mutations in LRP5.

Sclerostin might have additional roles during postnatal life. Sclerosteosis patients are unusually tall (Van Hul et al. (2001) European Journal of Radiology 40 198) suggesting a putative role for sclerostin in cartilage biology. Furthermore sclerostin is expressed in the kidney implying that it might play a yet uncharacterized role in this organ (Balemans et al. 2001 Hum Mol. Genet. 10(5):537, Balemans and Van Hul (2002) Developmental Biology 250, 231). Finally it has been shown to be expressed at least during embryonic development in the cardiovascular system (van Bezooijen et al. (2006) Dev Dyn. 2006; 236(2):606).

Versican (CSPG2)

Versican is a member of the lectican family that includes aggrecan, neurocan and brevican. It is a large chondroitin sulfate proteoglycan. Its N-terminal globular domain (G1) binds to the glycosaminoglycan hyaluronan, and its carboxy-terminal globular domain (G3) consists of two EGF-like domains, a lectin-like domain, and a complement regulatory protein-like domain. Four splice variants (V0-V4) of versican are known (Wight, T N (2002) Curr. Opin. Cell Biol.; 14 (5):617-23). Versican V0 and V1 are cleaved by ADAMTS1 and 4 (aggrecanase-1) (Sandy, et al. (2001) J. Biol. Chem.; 276 (16):13372-8) (Russell, et al. (2003) J. Biol. Chem.; 278 (43):42330-9) and versican V2 by ADAMTS4 (Westling, et al. (2004) Biochem. J.; 377 (Pt. 3):787-95). Versican has a wide tissue distribution. Nakamura et al studied versican expression in developing mandibles and hind limbs. Based on their results, they propose the following. Versican is expressed before differentiation of osteoblasts and is localized in osteoid during intramembranous ossification. In endochondral ossification, versican is expressed in periosteal cells overlying the active osteogenic region on the surface of calcified cartilage. Thereafter, bones undergoing either type of ossification proceed with the common sequential process of bone development. The bone matrix expands and woven bone rich in versican is formed. Versican mRNA and protein are detected in osteoblasts, in confined population of osteocytes as well as in bone matrix. As woven bone is altered into lamellar bone, versican expression in the bone matrix is decreased. The temporal and spatial mRNA expression pattern of ADAMTS1, 4 and 5 is comparable to that of versican. They hypothesized that osteoblasts and osteocytes may be involved in both production and degradation of versican by secreting ADAMTSs (Nakamura, et al. (2005) J. Histochem. Cytochem.; 53 (12):1553-62). Finally, versican and BMP signaling are linked as shown by the fact that BMP-2 decrease by half versican gene expression in rat intervertebral discs cells (Li, et al. (2004) J. Spinal Disord. Tech.; 17(5):423-8).

As described in further detail herein, versican (CSPG, IPI00009802.1) has been identified as a binding partner of sclerostin in the Hek293 cell culture supernatant fraction of a Tandem Affinity Purification binding assay. Versican is thought to be an important binding partner of sclerostin, or is part of a multi-complex consisting of at least sclerostin, the interaction between which is capable of inducing a refolding of sclerostin into a more stable conformation (thereby modulating sclerostin action). The sclerostin:versican interaction also serves as the nexus between sclerostin and Wnt signaling.

FREM 2

Frem2, encoded by the my gene (myelencephalic blebs gene), is a proposed ECM component, related to Fras1 and Frem1 (Fras1 related extracellular matrix) and considered to be orthologous to the sea urchin ECM3 protein. Its predicted protein arrangement consists of an N-terminal signal peptide followed by 13 tandemly arranged chondroitin sulfate proteoglycan domains (CSPG), 5 tandemly arranged CALXβ domains, a transmembrane helix and a short cytoplasmic tail with a consensus PDZ interaction motif.

A missense mutation in Frem2 has been detected in blebbing mutant (my^{UcI}) and two individuals with Fraser Syndrome, a multisystem malformation usually comprising cryptophthalmos, cutaneous syndactyly, ear abnormalities, renal agenesis and congenital heart defects. Interestingly, my^{UcI} mice display cutaneous syndactyly occasionally accompanied by bony syndactyly and polydactyly. The nucleotide transition 5914G->A, which results in the amino acid substitution E1972K, has been identified in two unrelated families. This mutation occurs in the second of five consecutive CALXβ domains and substitutes a residue that is conserved in all known CALXβ domains. Sequence similarity searches showed that CALXβ is related to cadherin domains, known to intercalate calcium ions in a negatively charged pocket between consecutive domains. Sequence to structure alignments have shown that Glu1972 is located in the Ca²⁺ binding pocket at the interface of CALXβ domains 2 and 3 and corresponds to a conserved position directly involved in the coordination of Ca2+. This suggests that calcium binding in the CALXβ-cadherin motif is important for normal functioning of FREM2 (Jadeja S, et al. (2005) Nat. Genet.; 37 (5):520-5).

As described in further detail herein, Frem2 (IPI00180707.7) has been identified as a binding partner of sclerostin, in the membrane purification fraction of Hek293 cells of a Tandem Affinity Purification binding assay. Frem2 is thought to be an important binding partner of sclerostin, or is part of a multi-complex consisting of at least sclerostin, the interaction between which can induce a refolding of sclerostin into a more stable conformation (thereby modulating sclerostin action).

Fibrillin 2 (FBN2)

The glycoproteins fibrillin-1 and 2 are major structural components of the extracellular calcium-binding microfibrils with average diameter of 10 nm. Fibrillins share a conserved multidomain structure with a high degree of amino acid sequence homology. Fibrillin-2 is composed of 47 EGF-like domain, 43 of which have consensus calcium binding sequences, interrupted by 8-cysteine containing modules, which are also found in latent TGF-131 binding protein (LTBP), a unique C- and N-terminal domains and a small glycine-rich domain. Fibrillin-2 is preferentially localized to elastic tissues, such as the elastic cartilage and the tunica media layer of the aorta (Putnam, et al. (1995) Nat. Genet.; 11 (4):456-8). In bone, fibrillin-2 mRNA, together with fibrillin-1, is abundantly expressed by human trabecular bone derived from the proximal femur and by mature bone-derived human osteoblasts in primary culture (Kitahama, et al. (2000) Bone; 27 (1):61-7).

Fibrillin-2 mutations result in congenital contractual arachnodactyly (CCA), an autosomal dominant disorder that is characterized by arachnodactyly, dolichostenomelia, scoliosis, multiple congenital contractures and abnormalities of the external ears. CCA is phenotypically similar to Marfan syndrome, resulting from a mutation in fibrillin-1, but does not effect the aorta and the eyes. FBN2 missense mutations cause substitution of distinct cysteine residues in separate EGF-like repeats in two patient with CCA (Putnam, et al 1995). Fibrillin-2-null mice exhibit a limb-patterning defect in the form of bilateral syndactyly, primarily due to defective mesenchyme differentiation. Syndactyly is associated with a disorganized matrix, but with normal BMP gene expression. Mice double heterozygous for null Fbn2 and Bmp7 alleles display the combined digit phenotype (syndactyly and polydactyly of both nullzygotes (Arteaga-Solis, et al. (2001) J. Cell Biol.; 154 (2):275-81.) Because polydactyly is a feature of homozygous, not hererozygous, BMP-7 null mice and because heterozygous fibrillin-2 mice are normal, the phenotype of the double heterozygous mice suggested functional interaction between fibrillin-2-rich microfibrils and BMP-7 signaling during limb patterning.

Fibrillin-2 may provide the structural scaffold that arranges morphogenetic clues in the intercellular space of the developing organism. This function could be exerted by binding directly to inactive growth factors (such as in the case of the latent TGF-β complex), indirectly through interaction with other matrix components (such as proteoglycans), or by a combination of both mechanisms (Arteaga-Solis, supra). Recently, BMP-7 was shown to co-localize with fibrillin-2 in fibrillin-1 null mice. (Gregory, et al. (2005) J. Biol. Chem.; 280 (30): 27970-80).

As described in further detail herein, fibrillin 2 precursor (FBN2, IPI00019439.1) has been identified as a binding partner of sclerostin in the membrane purification fraction of Hek293 cells in a Tandem Affinity Purification binding assay. Fibrillin 2 is thought to be an important binding partner of sclerostin, or is part of a multi-complex consisting of at least sclerostin. This binding can induce a refolding of sclerostin into a more stable conformation, and can initiate the functional interaction between fibrillin-2 and BMP signaling, thereby forming a link between sclerostin and BMP signaling. Likewise, the sclerostin-fibrillin 2 interaction can initiate the functional interaction between fibrillin-2 and Wnt signaling, thereby providing the nexus between sclerostin and Wnt signaling.

C6orf93

C6orf93 is an hypothetical protein, also named LTV1 homolog (S. cerevisiae). In human, it has been first described in 2002 in the frame of the NIH MGC Program (Strausberg, et al. (2002) PNAS; 99 (26):16899-903). In Saccharomyces cerevisiae, the low-temperature viability protein LTV1 encodes a non-essential, non-ribosomal protein. Strains lacking LTV1 and YAR1 display a hypersensitivity to various environmental stress, like osmotic and oxidative stress, low and high temperature and the presence of certain protein synthesis inhibitors revealing an unknown link between ribosome biogenesis factors and environmental stress sensitivity (Loar, et al. (2004) Genetics.; 168 (4):1877-89). Ltv1 interacts genetically with the gene for the small ribosomal subunit export factor Yrb2, suggesting that Ltv1 functions as one of several possible adapter proteins that link the nuclear export machinery to the small subunit (Seiser, et al. (2006) Genetics.; 174(2):679-691).

As described in further detail herein, C6orf93 (IPI0053032.1) has been identified as a binding partner of sclerostin in the Hek293 and UMR106 membrane purification fraction of a Tandem Affinity Purification binding assay. C6orf93 is thought to be an important binding partner of sclerostin, or is part of a multi-complex consisting of at least sclerostin. The sclerostin:C6orf93 interaction can induce a refolding of sclerostin into a more stable conformation (thereby modulating sclerostin action), and initiate a functional interaction between C6orf93 and BMP or Wnt signaling, and serves as a link between sclerostin and thhese pathways. The sclerostinC6orf93 interaction is also implicated in the sensitivity of osteocytes to environmental stress.

Syndecan-4 (Sdc4)

Syndecan-1 through -4 are single-pass integral membrane components, members of the heparan sulfate proteoglycan family. Each syndecan has a short cytoplasmic domain, a single-span transmembrane domain and an extracellular domain with attachment sites for three to five glycosaminoglycans chains, allowing them to directly connect the pericellular milieu with the cellular interior. Syndecan-4, also named amphighlycan or ryudocan, is a unique member because of its ability to activate intracellular signaling cascades and to form focal adhesion sites in mammalian cells. It binds fibronectin through its glycosaminoglycans chains, activates PKCα and the small GTPase RhoA, and together with integrins stabilizes focal adhesion sites (Tkachenko E, et al. (2005) Circ. Res.; 96 (5):488-500). In Xenopus, fibronectin regulates the ability of syndecan-4 to translocate Dsh to the plasma membrane, a landmark in the activation of non-canonical Wnt signaling (Munoz R, et al. (2006) Nat. Cell Biol.; 8 (5):492-500).

Syndecan-4 is ubiquitously expressed in several cell types, including primary rat calvarial osteoblasts where its mRNA expression is upregulated by FGF2 treatment. This upregulation is not an immediate response as suggested by the reduction of syndecan-4 mRNA following cycloheximide treatment. Osteoblast proliferation and mineralization, as well as ERK activation, are also enhanced by FGF2, but specifically diminished by anti-syndecan-4 antibody pretreatment (Song S J, et al. (2007) J. Cell Biochem.; 100(2):402-411). In C2C12 cells, syndecan-2 and -3 are upregulated by BMP-2 (Gutierrez J, et al. (2006) J. Cell Physiol.; 206 (1):58-67), however syndecan-3 is a negative modulator of BMP-2 signaling during chondrogenesis (Fisher M C, et al. (2006) Matrix Biol.; 25 (1):27-39). In Drosophila, syndecan localizes to developing axons, interacts genetically and physically with SLIT and Robo and promotes axonal and myotube guidance via SLIT/Robo signaling (Johnson K G, et al. (2004) Curr. Biol.; 14 (6):499-504) (Steigemann P, et al. (2004) Curr. Biol.; 14 (3):225-30). Finally, syndecan-4 can induce filopodia-like structures in activated B lymphocytes when seeded on syndecan-4 antibodies (Yamashita Y, et al. (1999) J. Immunol.; 162 (10):5940-8).

As described in further detail herein, syndecan-4 (sdc4, IPI00199629.1) has been identified as a binding partner of sclerostin in the rat osteosarcoma UMR106 cell culture supernatant fraction of a Tandem Affinity Purification binding assay. Syndecan is thought to be an important binding partner of sclerostin (defined herein as “a sclerostin-binding-partner”), or is part of a multi-complex consisting of at least sclerostin, SLIT, and syndecan-4. This binding can modulate the initiation of osteocyte dendrite-like processes, or induce a refolding of sclerostin into a more stable conformation and hence regulate sclerostin action. Furthermore, syndecan-4 can serve as a link between sclerostin and Wnt and/or BMP signaling.

SLIT2

SLLIT2 is a secreted protein which acts as molecular guidance cue in cellular migration, and its function is mediated by interaction with roundabout homolog receptors. It is expressed in the spinal cord and is involved in early body axis formation and cell patterning of the neural-tube.

Gremlin and Dan, which are structurally related to sclerostin, physically and functionally interact with Slit1 and Slit2 proteins and act thereby as inhibitors of monocyte chemotaxis (Chen et al. (2004) J. Immunol.; 173(10):5914). Furthermore Slit proteins are high-affinity ligands of the heparan sulfate proteoglycan glypican-1 (Ronca et al. (2001) J Biol. Chem. 276(31):29141), which was also identified in experiments described in the present invention as a sclerostin interaction partner. Furthermore syndecan, also described in the present invention, promotes axonal and myotube guidance by slit/robo signaling, (Johnson K G, et al. (2004) Curr. Biol. 14 (6):499-504) (Steigemann P, et al. (2004) Curr. Biol. 14 (3):225-30)

SLIT2 (IPI00006288.1) has been identified as a binding partner of sclerostin in the HEK293 cell culture membrane fraction of a Tandem Affinity Purification binding assay. SLIT2 is thought to be an important binding partner of sclerostin (defined herein as “a sclerostin-binding-partner”), or is part of a multi-complex consisting of at least sclerostin, SLIT, and possibly syndecan-4 and glypican 1. This binding can modulate the generation of the osteocyte dendrite-like processes, or induce a refolding of sclerostin into a more stable conformation and hence regulate sclerostin action. Furthermore, SLIT2 can serve as a link between sclerostin and Wnt and/or BMP signaling.

Glypican1 (Gpc1)

Glypicans modulate encounters of extracellular protein ligands with their receptors acting as co-receptors. They are known to modulate Wnt signaling (Capurro et al. (2005), Cancer Res.; 65(14):6245) and BMP signaling [for example by interacting with the BMP antagonists (Paine-Saunders et al. (2000) Dev Biol.; 225(1):179). For example mutations in glypican 3 result in various syndromes which are associated with bone overgrowth. For example. Simpson-Golabi-Behmel overgrowth syndrome (Pilia et al. (1996), Nat. Genet. 12(3):241) is the result of loss of Gpc3 control on Wnt signaling ((Song et al. (2005); Biol Chem 280(3):2116)).

Glypicans are expressed by cells of the osteoblastic lineage and have been suggested as potential modulators of bone remodeling (Sheu et al. (2002) J Bone Miner Res. 17 (5):915).

Furthermore glypican 1 binds to SLIT (Ronca et al. (2001) J Biol. Chem. 276(31):29141), which is also described in the present invention as a sclerostin interaction partner.

Gpc1 (IPI00137336.1) has been identified as a binding partner of sclerostin in the osteoblastic UMR-106 cell culture supernatant fraction of a Tandem Affinity Purification binding assay. Gpc1 is thought to be an important binding partner of sclerostin (defined herein as “a sclerostin-binding-partner”), or is part of a multi-complex consisting of at least sclerostin, Gpc1 and possibly syndecan-4 and SLIT2. This binding can modulate the generation of the osteocyte dendrite-like processes, or induce a refolding of sclerostin into a more stable conformation and hence regulate sclerostin action. Furthermore, glypican1 can serve as a link between sclerostin and Wnt and/or BMP signaling.

Agrin (AGRN)

Agrin is a large extracellular matrix heparin sulfate proteoglycan, with a molecular weight of about 600 kDa (200 kDa protein core). Alternative messenger RNA splicing generates an isoform encoding a cleaved signal sequence followed by the amino-terminal-agrin domain resulting into a secreted form of agrin (NtA-agrin) and an isoform containing a shorter amino terminus with an internal, non-cleaved signal peptide, converting the protein to a type II transmembrane protein (TM-agrin). Both isoforms are differentially expressed: NtA-agrin being ubiquitously expressed in most basal laminae-containing tissue and TM-agrin being preferentially expressed in the central nervous system (Burgess, et al. (2000) J. Cell Biol.; 151 (1):41-52) (Neumann, et al. (2001) Mol. Cell Neurosci.; 17 (1):208-25). Recently, it was shown that agrin is expressed in mouse chondrocytes and localizes to the growth plate (Hausser et al. (2007) Histochem Cell Biol.; 127:363). NtA-agrin-deficient mice have provided the evidence that agrin is required for the aggregation of acetylcholine receptors during postsynaptic development at the neuromuscular junction in skeletal muscle (Gautam, et al. (1996) Cell.; 85 (4):525-35). This ability requires the muscle-specific receptor tyrosine kinase MuSK, as demonstrated by the similarity of the agrin- and MuSK-deficient mice phenotypes (DeChiara, et al. (1996) Cell.; 85 (4):501-12). Overexpression of agrin in rat skeletal muscle cells induces formation of filopodia (Uhm, et al. (2001) J. Neurosci.; 21 (24):9678-89). Antibody-induced clustering of endogenous TM-agrin leads to increased formation of filopodia-like processes along axons of central and peripheral neurons (Annies, et al. (2006) Mol. Cell Neurosci.; 31 (3):515-24). Overexpression and downregulation via siRNA of TM-agrin in hippocampal neuron cultures suggest that TM-agrin positively regulates the number of filopodia on developing neuritis by its effect on both initiation and stabilization of filopodia (McCroskery, et al. (2006) Mol. Cell Neurosci.; 33(1):15-28).

As described in further detail herein, Agrin (IPI00374563.2) has been identified as a binding partner of sclerostin in the Hek293 cell culture supernatant fraction of a Tandem Affinity Purification binding assay. Agrin is thought to be an important binding partner of sclerostin, or is part of a multi-complex consisting of at least sclerostin, the interaction between which is capable of modulating the initiation and stabilization of osteocyte filopodia-like processes, or inducing a refolding of sclerostin into a more stable conformation (thereby modulating sclerostin action).

Serpine-2 (PN-1)

Serpine 2 encodes serpin peptidase inhibitor, also named protease nexin I (PN-1) or glia derived nexin precursor (PI7). It is a secreted protein of 43 kDa, member of the serine protease inhibitor (SERPIN) superfamily and has been described to be synthesized by astrocytes, smooth muscle, endothelial cells, and fibroblasts (Scott, et al. (1985) J. Biol. Chem.; 260 (11):7029-34) (Rosenblatt, et al. (1987) Brain Res.; 415 (1):40-8) (Festoff, et al. (1991) J. Cell Physiol.; 147 (1):76-86) (Bouton, et al. (2003) Arterioscler. Thromb. Vasc. Biol.; 23 (1):142-7). It is a potent inhibitor of thrombin and urinary plasminogen activator (uPA) and is a less potent but still effective inhibitor of plasmin and trypsin (Scott, et al. (1985) DNA Cell Biol.; 22 (2):95-105). The efficient catabolism of thrombin-PN-1 complexes is a synergistic mechanism that requires both LRP-1 and heparins: first the thrombin-PN-1 complexes are concentrated to the cell surface by heparins and subsequently internalized by LRP-1, before being degraded by the cells (Knauer, et al. (1997) J. Biol. Chem.; 272 (46):29039-45, Scott, et al. (2003) DNA Cell Biol.; 22(2):95-105).

In mouse embryonic fibroblasts, an alternative internalization of PN-1 complexes is mediated by syndecan-1 and activates the Ras-ERK signaling pathway. Free PN-1 can also be internalized (Li, et al. (2006) J. Cell Biochem.; 99 (3):936-51). PN-1 regulates vascular smooth muscle cell adhesion, spreading and migration (Richard, et al. (2006) J. Thromb. Haemost.; 4 (2):322-8). PN-1 expression is up-regulated in human skeletal muscle by injury-related factors like TNFalpha, TGFbeta and IL-1 (Mbebi, et al. (1999) J. Cell Physiol.; 179 (3):305-14). PN-1 has also been reported to be involved in neurite extension by inhibiting thrombin (Farmer, et al. (1990) Dev. Neurosci.; 12 (2):73-80). Finally, in NIH3T3 cells, PN-1 was shown to be a target gene of Prx2 known to be required for correct skeletogenesis (Scott, et al. (2003) DNA Cell Biol.; 22(2):95-105).

As described in further detail herein, PN-1 (IPI00203479.3) has been identified as a binding partner of sclerostin in the UMR106 cell culture supernatant fraction of a Tandem Affinity Purification binding assay. PN-1 is thought to be an important binding partner of sclerostin, or is part of a multi-complex consisting of at least sclerostin, the interaction between which is capable of induce a refolding of sclerostin into a more stable conformation (thereby modulating sclerostin action). The sclerostin:PN-1 interaction is also implicated in osteocyte outgrowth or sclerostin internalization and degradation.

Low-Density Lipoprotein Receptor-Related Protein 2 (LRP2, Megalin)

The low-density lipoprotein receptor family is a class of highly conserved cell surface receptors with broad function in cargo transport, internalization of macromolecules from the cell surface and cellular signaling. Megalin is a multiligand epithelial endocytic receptor, which is well characterized in the adult kidney and ileum where they form a complex essential for protein, lipid and vitamin uptake. It is also expressed on the apical surfaces of epithelial cells lining specific regions of the male and female reproductive tracts and in seminal vesicle (rat) where it acts as an endocytic receptor for seminal vesicle. Megalin knockout mice develop vitamin D deficiency and bone disease owing to an inability of the proximal tubules in the kidneys to capture the DBP/25-(OH)D3 complexes from the glomerular filtrate (Willnow et al. (1996) Proc. Natl. Acad. Sci. USA 93, 8460). In the same way, kidney-specific megalin knockout mice have severe plasma vitamin D deficiency, hypocalcaemia and serious bone disease, like the complete megalin knockout mice (Leheste et al. (2003) FASEB J. 17(2):247. Their skeleton is characterized by a decrease in bone mineral content, an increase in osteoid surfaces, and a lack of mineralizing activity. These features are consistent with osteomalacia as a consequence of hypovitaminosis D and demonstrate the crucial importance of the megalin pathway for systemic calcium homeostasis and bone metabolism.

LRP2 (IPI00024292.1) has been identified as a binding partner of sclerostin in the human embryonic kidney Hek293 membrane purification and supernatant fraction of a Tandem Affinity Purification binding assay. LRP2 is thought to be an important binding partner of sclerostin, or is part of a multi-complex consisting of at least sclerostin. Sclerostin is expressed in the kidney (Balemans and Van Hul (2002) Developmental Biology 250, 231). Consequently LRP2 could interact with sclerostin in the kidney modulating there its yet uncharacterized action. Furthermore LRP2 could be involved in sclerostin internalization in bone and subsequent degradation, modulating its action.

Low-Density Lipoprotein Receptor-Related Protein 4 (LRP4, Also Known as Megf7)

Megf7 is a member of the low-density lipoprotein receptor family. LRP4-deficient mice display polysyndactyly of the fore and hind limbs. Syndactyly is also a typical feature of abnormal sclerostin levels (both absence of sclerostin in sclerosteosis patients and the overexpression of sclerostin in mice results in syndactyly). Both LRP4 and sclerostin proteins play a role in apical ectodermal ridge (AER) formation and are expressed at embryonic day 9.5. Furthermore, both sclerostin and LRP4 can antagonize canonical Wnt signaling. (Johnson E B, et al. (2005) Hum Mol. Genet. 14(22):3523) (Simon-Chazottes D, et al. (2006) Genomics 87(5):673) (Loots G G, et al. (2005) Genome Res. 15(7):928-35)

As described in further detail herein, LRP4 (IPI00306851.3) has been identified as a binding partner of sclerostin in the membrane fraction of UMR-106 and Hek293 cells of a Tandem Affinity Purification binding assay. LRP4 is thought to be an important binding partner of sclerostin, or is part of a multi-complex consisting of at least sclerostin, the interaction between which is capable of induce a refolding of sclerostin into a more stable conformation (thereby modulating sclerostin action). Furthermore, LRP4 has been shown to act as an enhancer of sclerostin action in its role as a Wnt signaling inhibitor.

Low-Density Lipoprotein Receptor-Related Protein 6 (LRP6)

LRP6 is essential for the Wnt/beta catenin signaling pathway, by acting as a co-receptor together with Frizzled for Wnt. LRP6 binds DKK1 with high-affinity. This interaction with DKK1 blocks LRP6-mediated Wnt/beta catenin signaling. It has been recently shown that sclerostin—like DKK1—acts in vitro as a binding partner to LRP6 and thereby inhibits Wnt signaling (Semenov et al. (2005) J Biol. Chem. 280(29):26770).

LRP6 (IPI00000203.1) has been identified as a binding partner of sclerostin in the Hek293 and osteoblastic UMR106 cell culture membrane fraction of a Tandem Affinity Purification binding assay. These findings suggest that sclerostin exerts part of its action in vivo via LRP6 interaction. This also emphasizes the relevance of the findings from the Tandem Affinity Purification binding assay experiments, and therefore is viewed as a positive control.

Tenascin C

Tenascin-C is a large multimeric extracellular matrix protein of 240 kDa, including heptad repeats, EGF-like repeats, fibronectin type III domains, and a C-terminal globular domain shared with fibrinogens. Tenascins are primarily synthesized by cells in connective tissues (Chiquet-Ehrismann R (2004) Int. J. Biochem. Cell Biol.; 36 (6):986). They are classified as adhesion-modulating proteins because, contrary to other extracellular matrix proteins, tenascins promote only weak cell attachment and cell spreading is limited (Orend G and Chiquet-Ehrismann R (2000) Exp. Cell Res.; 261 (1):104).

Tenascin-C can impact several intracellular signaling molecules, like FAK, RhoA, cGMP-dependent protein kinase and 14-3-3tau. It can also directly bind to and activate the EGF receptor (Chiquet-Ehrismann R and Tucker R P (2004) Int. J. Biochem. Cell Biol.; 36 (6):1085). Tenascin-C supports differentiation of cultured osteoblast-like cells (Mackie E J and Ramsey S (1996) J. Cell Sci.; 109 (Pt 6):1597). In rat ulnae, immunohistochemical detection shows that only osteocytes within the new bone formed in response to load were strongly stained for tenascin-C. Osteocytes that had become embedded more recently, i.e., those closer to the periosteal surface, were unstained (Webb C M, et al (1997) J. Bone Miner. Res.; 12 (1):52). Tenascin-C influences integrin and syndecan signaling (Huang W, et al (2001) Cancer Res.; 61 (23):8586).

In hypertensive patients, tenascin-C is induced in response to mutated BMPR2s (Ihida-Stansbury K, et al (2006) Am. J. Physiol Lung Cell Mol. Physiol.; 291 (4):L694). Tenascin-C expression is suppressed by Wnt7a in high-density chick limb bud cell culture (Stott N S, Jiang T X and Chuong C M (1999) J. Cell Physiol.; 180 (3):314), and in chick embryo fibroblasts, TGFb induces tenascin expression. (Pearson C A, et al (1988) EMBO J.; 7 (10):2977). Tenascin-C increases neurite outgrowth from rat cerebellar granule neurons via interaction with integrin alpha7beta1 (Mercado M L, et al (2004) J. Neurosci.; 24 (1):238).

As described in further detail herein, tenascin-C (IPI00403938.1) has been identified as a binding partner of sclerostin in the UMR106 cell culture supernatant fraction of a Tandem Affinity Purification binding assay. Tenascin-C is thought to be an important binding partner of sclerostin, or is part of a multi-complex consisting of at least sclerostin. This interaction induce a refolding of sclerostin into a more stable conformation, and/or initiate the functional interaction between tenascin-C and BMP signaling, thereby providing a link between sclerostin and BMP signaling. Likewise, the interaction between tenascin-C and sclerostin initiate the functional interaction between tenascin-C and Wnt signaling, thereby providing a link between sclerostin and Wnt signaling. Moreover, it is involved in osteocytes outgrowth or a combination of these mechanisms.

Tripartite Motif (TRIM) Proteins (TRIM26, TRIM41)

The tripartite motif (TRIM) protein family is a an expanding family of RING (“really interesting new gene”) proteins, also known as RBCC proteins as they contain an RBCC motif, which comprises a RING domain, one or two B-boxes and a predicted coiled-coil region. TRIM/RBCC proteins are involved in a broad range of biological processes, including cell proliferation, differentiation, development, oncogenesis and apoptosis. The presence of the RING domain and its strong association to ubiquitination suggests a role for this protein family in the ubiquitination process. (Meroni G, et al. (2005) Bioessays. 27 (11):1147) (Nisole S, et al. (2005) Nat. Rev. Microbiol. 3 (10):799)

As described in further detail herein, TRIM26 (IPI00010948.2) has been identified in the Hek293 membrane fraction of a Tandem Affinity Purification binding assay and TRIM41 (IPI00414021.1) as a binding partner of sclerostin in the Hek293 and UMR106 membrane fraction of a Tandem Affinity Purification binding assay. TRIM26 and TRIM41 are thought to be important binding partners of sclerostin, or part of a multi-complex consisting of at least sclerostin, the interaction between which is capable of inducing sclerostin degradation. TRIM, together with LRP2 could be involved in sclerostin internalization and subsequent degradation.

IL-17 Receptor

The receptor for IL-17A (IL17RA) is a single-pass transmembrane protein of approximately 130 kDa and has an unusually large cytoplasmic tail. While the IL-17A cytokine is expressed only by T-cells, its receptor is ubiquitously expressed. As a consequence, IL-17 can act on a wide variety of cells to trigger expression of inflammatory effectors. Most of these effectors have been shown to have an impact on bone metabolism by either promoting osteoclastogenesis or exerting a bone protective effect (Gaffen S L (2004) Arthritis Res. Ther.; 6 (6):240).

Most IL-17-induced factors tend to be bone resorptive. For example, IL-6 has been shown to be a contributing factor to estrogen mediated bone loss (Jilka R L, et al (1992) Science.; 257 (5066):88). In mice overexpressing IL-17, bone erosion is mediated by RANKL (Lubberts E, et al (2003) J. Immunol.; 170 (5):2655). IL-17 is not involved in physiological regulation of bone homeostasis because of the absence of difference in bone mineral density, skeletal development as well as bone resorption and bone formation parameters in IL-17^{-/-} mice compared to wild type littermate. However, in an LPS-induced model of inflammatory bone destruction, the level of bone resorption was much less pronounced and the osteoclasts formation significantly reduced in IL-17^{-/-} mice compared to wild type mice, suggesting that Th17 cells are involved in the T cell-mediated osteoclastogenesis.

IL-17-mediated induction of RANKL and inflammatory cytokines, such as TNFα and IL-1, have been suggested to be involved in that process (Sato K, et al (2006) J. Exp. Med.; 203 (12):2673). IL-17 is also a potent inducer of neutrophil recruitment and activation, due in large part to its ability to promote chemokine secretion. Neutrophils are thought to contribute to bone destruction during chronic inflammation. However, neutrophils are generally considered to be bone protective in the context of periodontal disease-induced bone loss (Kantarci A, Oyaizu Z and Van Dyke T E (2003) J. Periodontol.; 74 (1):66). Signaling of IL-17RA is poorly defined. Pathways implicated might include the NF-KB pathway, the C/EBP family as well as MAPK and GSK3β involved in C/EBP phosphorylation, ERK1 and 2, JNK, p38 and PI-3K/Akt (Gaffen S L, et al (2006) Vitam. Horm.; 74:255-82.:255).

As described in further detail herein IL-17RA (IPI00304993.3) has been identified as a binding partner of sclerostin in the Hek293 membrane fraction of a Tandem Affinity Purification binding assay. IL-17RA, or IL-17RB, IL-17RC, IL-17RD and IL-17RE are thought to be important binding partners of sclerostin, or are part of a multi-complex consisting of at least sclerostin, the interaction between which is capable of inducing a refolding of sclerostin into a more stable conformation, and/or initiate the functional interaction between IL-17 receptor and BMP signaling, thereby providing a link between sclerostin and BMP signaling. Likewise, the interaction between IL-17 receptor and sclerostin initiate the functional interaction between IL-17 receptor and Wnt signaling, thereby providing a link between sclerostin and Wnt signaling. Moreover, it is involved in osteocytes outgrowth or a combination of these mechanisms.

Alkaline Phosphatase (ALPL)

In most mammals, there are four different isozymes: placental, placental-like, intestinal and tissue non-specific (liver/bone/kidney, ALPL). Defects in alkaline phosphatase liver/bone/kidney (ALPL) are a cause of hypophosphatasia infantile, an inherited metabolic bone disease characterized by defective skeletal mineralization, suggesting that ALPL plays a role in skeletal mineralization (Fedde K N et al. (1999) JBMR 14(12):2015-2026).

ALPL is a bone formation marker. During osteogenesis, alkaline phosphatase is not detected in osteoprogenitors. It's expression starts once the proliferative capacity of the preosteoblasts colonies is lost and nodule formation initiated. Expression is maintained from that time point onwards (Liu et al. (1994) Devel Biol. 166:220-234). Several BMPs have been described to induce alkaline phosphatase in osteoblasts-like cells (Cheng H et al. (2003) J Bone Joint Surg Am. 85:1544-1552). In addition, Rawadi G et al. (2003) showed that BMP-2 controls alkaline phosphatase expression by a Wnt autocrine loop (JBMR 18(10):1842-1853).

As described in further detail herein, ALPL (IPI00327143.1) has been identified as a binding partner of sclerostin in the UMR106 cell culture supernatant fraction of a Tandem Affinity Purification binding assay. ALPL is thought to be an important binding partner of sclerostin, or is part of a multi-complex consisting of at least sclerostin, Furthermore, SOST has been shown to directly inhibit ALPL enzymatic activity in a cell-free based assay.

Screening Assays

The invention provides methods (also referred to herein as “screening assays”) for identifying modulators, e.g, candidate or test compounds or agents (an antibody, an Antibody-like Scaffold, a small molecule, fusion protein, peptide, mimetic, or inhibitory nucleotide (e.g., RNAi)) which bind to sclerostin or a sclerostin-binding-partner, or to protein members of related complexes and have a stimulatory or inhibitory effect on, for example, sclerostin expression or activity.

Said methods include methods for identifying candidate or test compounds or agents capable of modulating the sclerostin:sclerostin-binding-partner interaction, which method comprises measuring the alteration of sclerostin interaction with a sclerostin-binding-partner occasioned by said agent. Preferably said method comprises the steps of: a) contacting sclerostin with a sclerostin-binding-partner in the presence and absence of a test agent under conditions permitting the interaction of the sclerostin-binding-partner with sclerostin; and b) measuring interaction of the sclerostin-binding-partner with sclerostin in both the presence and absence of said test agent wherein (i) a decrease in sclerostin:sclerostin-binding-partner interaction in the presence of the test agent, relative to the interaction in the absence of the test agent, identifies the test agent as an agonist of the sclerostin:sclerostin-binding-partner interaction, and wherein (ii) an increase in the interaction in the presence of the test agent, relative to the interaction in the absence of the test agent, identifies the test agent as an antagonist of the sclerostin:sclerostin-binding-partner interaction.

Inhibition of the sclerostin:sclerostin-binding-partner interaction occurs in the case of an antagonist, inhibitor, negative modulator, or negative regulator of sclerostin or a sclerostin-binding-partner. The antagonist has the effect of reducing or completely blocking the binding of the sclerostin-binding-partner to sclerostin. The antagonist may decrease the binding of sclerostin to a sclerostin-binding-partner by at least 10%, 20%, 30%, 40%, 50%, 60%, 70%, 80%, 90% in the presence of antagonist, as compared to the binding in the absence of antagonist, or by an amount in the range between any two of the aforementioned values. Preferably, the antagonist decreases said binding by at least 10%. The binding can be determined by, for example, measuring the binding constant using biochemical and/or biophysical methods as described herein.

In one embodiment, the present invention provides methods for identifying an agent capable of modulating the sclerostin:Frem2 interaction, which method comprises measuring the alteration of sclerostin binding to Frem2 occasioned by said agent. In another embodiment, the present invention provides methods for identifying an agent capable of modulating the sclerostin:Versican (CSPG2) interaction, which method comprises measuring the alteration of sclerostin binding to Versican occasioned by said agent. In yet another embodiment, the present invention provides methods for identifying an agent capable of modulating the sclerostin:Fibrillin 2 (FBN2) interaction, which method comprises measuring the alteration of sclerostin binding to Fibrillin 2 occasioned by said agent. In yet another embodiment, the present invention provides methods for identifying an agent capable of modulating the sclerostin:C6orf93 interaction, which method comprises measuring the alteration of sclerostin binding to C6orf93 occasioned by said agent. In another embodiment, the present invention provides methods for identifying an agent capable of modulating the sclerostin:Syndecan-4 (Sdc4) interaction, which method comprises measuring the alteration of sclerostin binding to Syndecan-4 occasioned by said agent. In still another embodiment, the present invention provides methods for identifying an agent capable of modulating the sclerostin:Agrin (AGRN) interaction, which method comprises measuring the alteration of sclerostin binding to Agrin occasioned by said agent. In still another embodiment, the present invention provides methods for identifying an agent capable of modulating the sclerostin:Serpine-2 (PN-1) interaction, which method comprises measuring the alteration of sclerostin binding to Serpine-2 occasioned by said agent.

In still another embodiment, the present invention provides methods for identifying an agent capable of modulating the sclerostin:SLIT2 interaction, which method comprises measuring the alteration of sclerostin binding to SLIT2 occasioned by said agent. In still another embodiment, the present invention provides methods for identifying an agent capable of modulating the sclerostin:Glypican1 interaction, which method comprises measuring the alteration of sclerostin binding to Glypican1 occasioned by said agent. In still another embodiment, the present invention provides methods for identifying an agent capable of modulating the sclerostin:LRP2 interaction, which method comprises measuring the alteration of sclerostin binding to LRP2 occasioned by said agent. In still another embodiment, the present invention provides methods for identifying an agent capable of modulating the sclerostin:LRP4 interaction, which method comprises measuring the alteration of sclerostin binding to LRP4 occasioned by said agent. In still another embodiment, the present invention provides methods for identifying an agent capable of modulating the sclerostin:LRP6 interaction, which method comprises measuring the alteration of sclerostin binding to LRP6 occasioned by said agent. In still another embodiment, the present invention provides methods for identifying an agent capable of modulating the sclerostin:Tenascin C interaction, which method comprises measuring the alteration of sclerostin binding to Tenascin C occasioned by said agent. In still another embodiment, the present invention provides methods for identifying an agent capable of modulating the sclerostin:TRIM26 interaction, which method comprises measuring the alteration of sclerostin binding to TRIM26 occasioned by said agent. In still another embodiment, the present invention provides methods for identifying an agent capable of modulating the sclerostin:TRIM41 interaction, which method comprises measuring the alteration of sclerostin binding to TRIM41 occasioned by said agent. In still another embodiment, the present invention provides methods for identifying an agent capable of modulating the sclerostin:IL17-R interaction, which method comprises measuring the alteration of sclerostin binding to IL17-R occasioned by said agent. In still another embodiment, the present invention provides methods for identifying an agent capable of modulating the sclerostin:ALPL interaction, which method comprises measuring the alteration of sclerostin binding to ALPL occasioned by said agent.

The candidate or test compound or agent can be an antibody, an antibody-like scaffold, a small molecule, fusion protein, peptide, mimetic, or inhibitory nucleotide (e.g., RNAi) directed against (i) sclerostin; (ii) the sclerostin-binding-partner; (iii) a novel site (e.g., a newly created epitopic determinant) created by the sclerostin:sclerostin-binding-partner interaction, or (iv) a protein complex comprising any of the same.

Alterations in sclerostin:sclerostin-binding-partner interaction, sclerostin or sclerostin-binding-partner protein activity, and/or sclerostin pathway activity may be measured by PCR, Taqman PCR, phage display systems, gel electrophoresis, reporter gene assay, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine. The alterations may also be measured by using a method selected from label displacement, surface plasmon resonance, fluorescence resonance energy transfer (FRET) or bioluminescence resonance energy transfer (BRET), fluorescence quenching, and fluorescence polarization.

The change in sclerostin or sclerostin-binding-partner protein activity and/or sclerostin pathway activity may be detected by detecting a change in the interaction between sclerostin:sclerostin-binding-partner, by detecting a change in the level of sclerostin or sclerostin-binding-partner, or by detecting a change in the level of one or more of the proteins in the sclerostin pathway. Cells in which the above-described may be detected can be of bone, mesenchymal, kidney (e.g., HEK), or hemopoietic origin, may be cultured cells, or may be obtained from or may be within a transgenic organism. Such transgenic organisms include, but are not limited to a mouse, rat, rabbit, sheep, cow or primate.

For screening experiments involving alterations in the sclerostin:sclerostin-binding-partner interaction, cells expressing sclerostin or sclerostin-binding-partners may be incubated in binding buffer with labeled sclerostin-binding-partner in the presence or absence of increasing concentrations of a candidate agent. To validate and calibrate the assay, control competition reactions using increasing concentrations of unlabeled sclerostin-binding-partner can be performed. After incubation, a washing step is performed to remove unbound sclerostin-binding-partner. Bound, labeled sclerostin-binding-partner is measured as appropriate for the given label (e.g., scintillation counting, fluorescence, antibody-dye etc.). A decrease of at least 10% (e.g., at least 20%, 30%, 40%, 50%, or 60%) in the amount of labeled sclerostin-binding-partner bound in the presence of candidate agent indicates displacement of binding by the candidate agent.

Candidate agent may be considered to bind specifically in this or other assays described herein if they displace at least 10%, 20%, 30%, 40%, 50%, 60% and preferably at least 10% of labeled sclerostin-binding-partner (sub-saturating sclerostin-binding-partner dose) at a concentration of 1 mM or less. Of course, the roles of sclerostin-binding-partner and sclerostin may be switched; the skilled person may adapt the method so sclerostin is applied to sclerostin-binding-partner in the presence of various concentrations of candidate agent to determine alterations in the sclerostin:sclerostin-binding-partner interaction.

Alterations of the sclerostin:sclerostin-binding-partner interaction can be monitored by surface plasmon resonance (SPR). Surface plasmon resonance assays can be used as a quantitative method to measure binding between two molecules by the change in mass near an immobilized sensor caused by the binding or loss of binding of sclerostin-binding-partner from the aqueous phase to sclerostin immobilized on the sensor. This change in mass is measured as resonance units versus time after injection or removal of the sclerostin-binding-partner or candidate agent and is measured using a Biacore Biosensor (Biacore AB). Sclerostin can be immobilized on a sensor chip (for example, research grade CM5 chip; Biacore AB) according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference.). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for sclerostin-binding-partner binding to sclerostin in an SPR assay can be fine-tuned by one of skill in the art using the conditions reported by Sarrio et al. as a starting point.

SPR can assay for inhibitors of binding in at least two ways. First, sclerostin-binding-partner can be pre-bound to immobilized sclerostin, followed by injection of candidate agent at a concentration ranging from 0.1 nM to 1 μM. Displacement of the bound sclerostin-binding-partner can be quantitated, permitting detection of inhibitor binding. Alternatively, the chip-bound sclerostin can be pre-incubated with candidate agent and challenged with sclerostin-binding-partner. A difference in sclerostin-binding-partner binding to sclerostin exposed to inhibitor relative to that on a chip not pre-exposed to inhibitor will demonstrate binding or displacement of sclerostin-binding-partner in the presence of inhibitor. In either assay, a decrease of 10% (e.g., 20%, 30%, 40%, 50%, 60%) or more in the amount of sclerostin-binding-partner bound in the presence of candidate agent, relative to the amount of a sclerostin-binding-partner bound in the absence of candidate agent that the candidate agent inhibits the interaction of sclerostin and sclerostin-binding-partner. While sclerostin is immobilized in the above, the skilled person may readily adapt the method so that sclerostin-binding-partner is the immobilized component.

Another method of detecting inhibition of binding of sclerostin-binding-partner to sclerostin uses fluorescence resonance energy transfer (FRET). FRET is a quantum mechanical phenomenon that occurs between a fluorescence donor (D) and a fluorescence acceptor (A) in close proximity to each other (usually <100 angstroms of separation) if the emission spectrum of D overlaps with the excitation spectrum of A. The molecules to be tested, e.g., sclerostin-binding-partner and sclerostin, are labeled with a complementary pair of donor and acceptor fluorophores. While bound closely together by the sclerostin:sclerostin-binding-partner interaction, the fluorescence emitted upon excitation of the donor fluorophore will have a different wavelength than that emitted in response to that excitation wavelength when the sclerostin-binding-partner and sclerostin are not bound, providing for quantitation of bound versus unbound molecules by measurement of emission intensity at each wavelength. Donor fluorophores with which to label the sclerostin are well known in the art. Of particular interest are variants of the A. victoria GFP known as Cyan FP(CFP, Donor (D)) and Yellow FP(YFP, Acceptor(A)). As an example, the YFP variant can be made as a fusion protein with sclerostin. Vectors for the expression of GFP variants as fusions (Clontech) as well as fluorophore-labeled sclerostin-binding-partner compounds (Molecular Probes) are known in the art.

The addition of a candidate agent to the mixture of labeled sclerostin-binding-partner and YFP-sclerostin will result in an inhibition of energy transfer evidenced by, for example, a decrease in YFP fluorescence relative to a sample without the candidate agent. In an assay using FRET for the detection of sclerostin:sclerostin-binding-partner interaction, a 10% or greater (e.g. equal to or more than 20%, 30%, 40%, 50%, 60%) decrease in the intensity of fluorescent emission at the acceptor wavelength in samples containing a candidate agent, relative to samples without the candidate agent, indicates that the candidate agent inhibits the sclerostin:sclerostin-binding-partner interaction. Conversely, a 10% or greater (e.g., equal to or more than 20%, 30%, 40%, 50%, 60%) increase in the intensity of fluorescent emission at the acceptor wavelength in samples containing a candidate agent, relative to samples without the candidate agent, indicates that the candidate agent induces a conformational change and enhance the sclerostin:sclerostin-binding-partner interaction.

A variation on FRET uses fluorescence quenching to monitor molecular interactions. One molecule in the interacting pair can be labeled with a fluorophore, and the other with a molecule that quenches the fluorescence of the fluorophore when brought into close apposition with it. A change in fluorescence upon excitation is indicative of a change in the association of the molecules tagged with the fluorophore:quencher pair. Generally, an increase in fluorescence of the labeled sclerostin is indicative that the sclerostin-binding-partner molecule bearing the quencher has been displaced. Of course, a similar effect would arise when sclerostin-binding-partner is fluorescently labeled and sclerostin bears the quencher. For quenching assays, a 10% or greater increase (e.g., equal to or more than 20%, 30%, 40%, 50%, 60%) in the intensity of fluorescent emission in samples containing a candidate agent, relative to samples without the candidate agent, indicates that the candidate agent inhibits sclerostin:sclerostin-binding-partner interaction. Conversely, a 10% or greater decrease (e.g., equal to or more than 20%, 30%, 40%, 50%, 60%) in the intensity of fluorescent emission in samples containing a candidate agent, relative to samples without the candidate agent, indicates that the candidate induces a conformational change and enhance the sclerostin:sclerostin-binding-partner interaction.

In addition to the surface plasmon resonance and FRET methods, fluorescence polarization measurement is useful to quantitate binding. The fluorescence polarization value for a fluorescently-tagged molecule depends on the rotational correlation time or tumbling rate. Complexes, such as those formed by sclerostin associating with a fluorescently labeled sclerostin-binding-partner, have higher polarization values than uncomplexed, labeled sclerostin-binding-partner. The inclusion of a candidate agent of the sclerostin:sclerostin-binding-partner interaction results in a decrease in fluorescence polarization, relative to a mixture without the candidate agent, if the candidate agent disrupts or inhibits the interaction of sclerostin with sclerostin-binding-partner. Fluorescence polarization is well suited for the identification of small molecules that disrupt the formation of complexes. A decrease of 10% or more (e.g., equal to or more than 20%, 30%, 40%, 50%, 60%) in fluorescence polarization in samples containing a candidate agent, relative to fluorescence polarization in a sample lacking the candidate agent, indicates that the candidate agent inhibits sclerostin:sclerostin-binding-partner interaction.

Another detection system is bioluminescence resonance energy transfer (BRET), which uses light transfer between fusion proteins containing a bioluminescent luciferase and a fluorescent acceptor. In general, one molecule of the sclerostin:sclerostin-binding-partner interacting pair is fused to a luciferase (e.g. Renilla luciferase (Rluc))—a donor which emits light in the wavelength of ˜395 nm in the presence of luciferase substrate (e.g. DeepBlueC). The other molecule of the pair is fused to an acceptor fluorescent protein that can absorb light from the donor, and emit light at a different wavelength. An example of a fluorescent protein is GFP (green fluorescent protein) which emits light at ˜510 nm. The addition of a candidate agent to the mixture of donor fused-sclerostin-binding-partner and acceptor-fused-sclerostin will result in an inhibition of energy transfer evidenced by, for example, a decrease in acceptor fluorescence relative to a sample without the candidate agent. In an assay using BRET for the detection of sclerostin:sclerostin-binding-partner interaction, a 10% or greater (e.g. equal to or more than 20%, 30%, 40%, 50%, 60%) decrease in the intensity of fluorescent emission at the acceptor wavelength in samples containing a candidate agent, relative to samples without the candidate agent, indicates that the candidate agent inhibits the sclerostin:sclerostin-binding-partner interaction. Conversely, a 10% or greater (e.g. equal to or more than 20%, 30%, 40%, 50%, 60%) increase in the intensity of fluorescent emission at the acceptor wavelength in samples containing a candidate agent, relative to samples without the candidate agent, indicates that the candidate agent induces a conformational change and enhance the sclerostin:sclerostin-binding-partner interaction.

It should be understood that any of the binding assays described herein can be performed with a non-sclerostin-binding-partner ligand (for example, agonist, antagonist, etc.) of sclerostin, e.g., a small molecule identified as described herein or sclerostin-binding-partner mimetics including but not limited to any of natural or synthetic peptide, a polypeptide, an antibody or antigen-binding fragment thereof, a lipid, a carbohydrate, and a small organic molecule.

Any of the binding assays described can be used to determine the presence of an inhibitor in a sample, e.g., a tissue sample, that binds to the sclerostin, or that affects the binding of sclerostin-binding-partner to sclerostin. To do so, sclerostin is reacted with sclerostin-binding-partner in the presence or absence of the sample, and binding is measured as appropriate for the binding assay being used. A decrease of 10% or more (e.g., equal to or more than 20%, 30%, 40%, 50%, 60%) in the binding of sclerostin-binding-partner indicates that the sample contains an inhibitor that modulates sclerostin-binding-partner binding to the sclerostin. The FRET and BRET binding assays described can also be used to determine the presence of an enhancer in a sample, e.g., a tissue sample, that binds to the sclerostin, or that affects the binding of sclerostin-binding-partner to sclerostin. To do so, sclerostin is reacted with sclerostin-binding-partner in the presence or absence of the sample, and binding is measured as appropriate for the binding assay being used. An increase of 10% or more (e.g., equal to or more than 20%, 30%, 40%, 50%, 60%) in the binding of sclerostin-binding-partner indicates that the sample contains an enhancer that modulates sclerostin-binding-partner binding to the sclerostin.

Any of the binding assays described can also be used to determine the presence of an inhibitor in a library of compounds. The FRET and BRET binding assays described can also be used to determine the presence of an enhancer in a library of compounds. Such screening techniques using, for example, high throughput screening are well known in the art.

The present invention also provides methods for identifying an agent capable of modulating the sclerostin:sclerostin-binding-partner interaction, which method comprises measuring the signaling response induced by the sclerostin:sclerostin-binding-partner interaction in the presence of said agent, and comparing it with the signaling response induced by the sclerostin:sclerostin-binding-partner interaction in the absence of said agent. Preferably, said method comprises the steps of: a) contacting sclerostin with a sclerostin-binding-partner in the presence and absence of a test agent under conditions permitting the interaction of the sclerostin-binding-partner with sclerostin; and b) measuring a signaling response induced by the sclerostin:sclerostin-binding-partner interaction, wherein a change in response in the presence of the test agent of at least 10% compared with the response in the absence of the test agent indicates the test agent is identified as capable of modulating the sclerostin:sclerostin-binding-partner interaction.

An increase in signaling response in the presence of the test agent of at least 10% compared with the response in the absence of the test agent identifies the test agent as an agonist of the sclerostin:sclerostin-binding-partner interaction. A decrease in signaling response in the presence of the test agent of at least 10% compared with the response in the absence of the test agent identifies the test agent as an antagonist of the sclerostin:sclerostin-binding-partner interaction.

Modulators of the sclerostin:sclerostin-binding-partner interaction (e.g., those identified by the methods of the invention) may change the signaling response induced by the sclerostin:sclerostin-binding-partner interaction by at least 10%, 20%, 30%, 40%, 50%, 60%, 70%, 80%, 90% in the presence of the modulator, as compared to the signaling in the absence of modulator, or by an amount in the range between any two of the aforementioned values. Preferably, the modulator changes said signaling by at least 10%. The change can be an increase or a decrease depending on the monitored activity. The signaling can be determined by methods well known in the art, such as for example, by measuring signaling levels using a reporter construct as described below.

The present invention provides methods for identifying an agent capable of modulating the sclerostin:Frem2 interaction, which method comprises measuring the signaling response induced by the sclerostin:Frem2 interaction in the presence of said agent, and comparing it with the signaling response induced by the sclerostin:Frem2 interaction in the absence of said agent. In one embodiment, the present invention provides methods for identifying an agent capable of modulating the sclerostin:Versican interaction, which method comprises measuring the signaling response induced by the sclerostin:Versican interaction in the presence of said agent, and comparing it with the signaling response induced by the sclerostin:Versican interaction in the absence of said agent. In another embodiment, the present invention provides methods for identifying an agent capable of modulating the sclerostin:Fibrillin 2 interaction, which method comprises measuring the signaling response induced by the sclerostin:Fibrillin 2 interaction in the presence of said agent, and comparing it with the signaling response induced by the sclerostin:Fibrillin 2 interaction in the absence of said agent. In yet another embodiment, the present invention provides methods for identifying an agent capable of modulating the sclerostin:C6orf93 interaction, which method comprises measuring the signaling response induced by the sclerostin:C6orf93 interaction in the presence of said agent, and comparing it with the signaling response induced by the sclerostin:C6orf93 interaction in the absence of said agent. In yet another embodiment, the present invention provides methods for identifying an agent capable of modulating the sclerostin:Syndecan-4 interaction, which method comprises measuring the signaling response induced by the sclerostin:Syndecan-4 interaction in the presence of said agent, and comparing it with the signaling response induced by the sclerostin:Syndecan-4 interaction in the absence of said agent. In still another embodiment, the present invention provides methods for identifying an agent capable of modulating the sclerostin:Agrin interaction, which method comprises measuring the signaling response induced by the sclerostin:Agrin interaction in the presence of said agent, and comparing it with the signaling response induced by the sclerostin:Agrin interaction in the absence of said agent. In yet another embodiment, the present invention provides methods for identifying an agent capable of modulating the sclerostin:Serpine-2 interaction, which method comprises measuring the signaling response induced by the sclerostin:Serpine-2 interaction in the presence of said agent, and comparing it with the signaling response induced by the sclerostin:Serpine-2 interaction in the absence of said agent.

In yet another embodiment, the present invention provides methods for identifying an agent capable of modulating the sclerostin:LRP2 interaction, which method comprises measuring the signaling response induced by the sclerostin:LRP2 interaction in the presence of said agent, and comparing it with the signaling response induced by the sclerostin:LRP2 interaction in the absence of said agent. In yet another embodiment, the present invention provides methods for identifying an agent capable of modulating the sclerostin:LRP4 interaction, which method comprises measuring the signaling response induced by the sclerostin:LRP4 interaction in the presence of said agent, and comparing it with the signaling response induced by the sclerostin:LRP4 interaction in the absence of said agent. In yet another embodiment, the present invention provides methods for identifying an agent capable of modulating the sclerostin:LRP6 interaction, which method comprises measuring the signaling response induced by the sclerostin:LRP6 interaction in the presence of said agent, and comparing it with the signaling response induced by the sclerostin:LRP6 interaction in the absence of said agent. In yet another embodiment, the present invention provides methods for identifying an agent capable of modulating the sclerostin:Glypican1 interaction, which method comprises measuring the signaling response induced by the sclerostin:Glypican1 interaction in the presence of said agent, and comparing it with the signaling response induced by the sclerostin:Glypican1 interaction in the absence of said agent. In yet another embodiment, the present invention provides methods for identifying an agent capable of modulating the sclerostin:SLIT2 interaction, which method comprises measuring the signaling response induced by the sclerostin:SLIT2 interaction in the presence of said agent, and comparing it with the signaling response induced by the sclerostin:SLIT2 interaction in the absence of said agent. In yet another embodiment, the present invention provides methods for identifying an agent capable of modulating the sclerostin:Tenascin C interaction, which method comprises measuring the signaling response induced by the sclerostin:Tenascin C interaction in the presence of said agent, and comparing it with the signaling response induced by the sclerostin:Tenascin C interaction in the absence of said agent. In yet another embodiment, the present invention provides methods for identifying an agent capable of modulating the sclerostin:IL17-R interaction, which method comprises measuring the signaling response induced by the sclerostin:IL17-R interaction in the presence of said agent, and comparing it with the signaling response induced by the sclerostin:IL17-R interaction in the absence of said agent. In yet another embodiment, the present invention provides methods for identifying an agent capable of modulating the sclerostin:TRIM26 interaction, which method comprises measuring the signaling response induced by the sclerostin:TRIM26 interaction in the presence of said agent, and comparing it with the signaling response induced by the sclerostin:TRIM26 interaction in the absence of said agent. In yet another embodiment, the present invention provides methods for identifying an agent capable of modulating the sclerostin: TRIM41 interaction, which method comprises measuring the signaling response induced by the sclerostin:TRIM41 interaction in the presence of said agent, and comparing it with the signaling response induced by the sclerostin:TRIM41 interaction in the absence of said agent. In yet another embodiment, the present invention provides methods for identifying an agent capable of modulating the sclerostin:ALPL interaction, which method comprises measuring the signaling response induced by the sclerostin:ALPL interaction in the presence of said agent, and comparing it with the signaling response induced by the sclerostin:ALPL interaction in the absence of said agent.

The signaling response is preferably the response of the Wnt and/or the BMP pathway, in which case an inhibitor would cause an increase in Wnt and/or BMP pathway activities. The signaling response can be determined, for example, measuring signaling levels using a reporter construct. For example, a suitable mammalian cell displaying a sclerostin-binding-partner or sclerostin may be transfected with a reporter construct comprising a promoter which is responsive to Wnt and/or BMP. When sclerostin binds a sclerostin-binding-partner, inhibiting the Wnt and/or BMP pathways, expression of a report protein is inhibited, which reduction can be measured, for example, by immunoassay, fluorescence, light measurement, etc., depending on the nature of the reporter protein. The expression is measured in the presence and absence of candidate agent.

By way of a specific example of a cell-based assay for measuring Wnt signaling, a reporter construct may be a Wnt/β-catenin dependent SuperTOPFlash (STF) luciferase reporter vector containing ten TCF-binding sites, driving the expression of firefly luciferase. This together with a Wnt expression vector (such as expression constructs for mouse Wnt1) and a Renilla expression vector (driven by SV40, used for normalization) may be transfected into Hek293 cells or any other suitable cell line. The transfected cell leads to activation of the STF-luciferase reporter, which activation can be blocked by sclerostin.

The present invention provides a method for identifying a sclerostin-binding-partner mimetic, which mimetic has the same, similar or improved functional effect as sclerostin-binding-partner interaction with sclerostin, wherein the method comprises measuring the interaction with sclerostin by a candidate mimetic. Preferably, said method comprises: a) contacting sclerostin with a candidate mimetic under conditions permitting the interaction of the mimetic with sclerostin; and b) measuring interaction of the mimetic with sclerostin, wherein the interaction is at least 10% of that observed for the various sclerostin:sclerostin-binding-partner interactions described herein, distinguishes the candidate mimetic as a sclerostin-binding-partner mimetic of the invention.

Furthermore, the present invention provides a method for identifying a sclerostin-binding-partner mimetic, which mimetic has the same, similar or improved functional effect as sclerostin-binding-partner interaction with sclerostin, wherein the method comprises measuring the signaling response induced by the sclerostin-mimetic interaction and comparing it with the signaling response induced by the sclerostin:sclerostin-binding-partner interaction. Preferably, said method comprises: a) contacting sclerostin with a candidate mimetic under conditions permitting the interaction of the mimetic with sclerostin; and b) measuring a signaling response induced by the sclerostin-mimetic interaction, wherein a signaling response that is at least 10% of that observed for the various sclerostin:sclerostin-binding-partner interactions described herein distinguishes the candidate mimetic as a sclerostin-binding-partner mimetic of the invention.

By way of non-limiting example, the present invention provides methods for identifying Frem2 or LRP4 mimetics that have the same, similar or improved functional effects as those of the interaction between Frem2 or LRP4 and sclerostin under normal physiological conditions.

A sclerostin-binding-partner mimetic is a compound that has the same, similar or improved functional effect as sclerostin-binding-partner binding to sclerostin. It may be a compound that contains an arrangement of functional groups often with additional hydrophobic or charged groups to resemble the active conformation of the binding region of the native sclerostin-binding-partner structure. It is to be understood that a sclerostin-binding-partner mimetic may also include a native sclerostin-binding-partner or its derivative.

According to one aspect of the invention, a mimetic exhibits at least 10%, 20%, 30%, 40%, 50%, 60%, 70%, 80%, 90% of the binding of a sclerostin-binding-partner for sclerostin or a value in the range between any two of the aforementioned values. Preferably, the mimetic exhibits at least 20% of the binding activity of sclerostin-binding-partner for sclerostin.

According to one aspect of the invention, a mimetic exhibits at least 10%, 20%, 30%, 40%, 50%, 60%, 70%, 80%, 90% of the signaling activity of a sclerostin-binding-partner or a value in the range between any two of the aforementioned values. Preferably, the mimetic exhibits at least 20% of the signaling activity of sclerostin-binding-partner.

The measuring of mimetic signaling activity of interaction with sclerostin can be performed by methods described herein for other assays, such as SPR and FRET.

According to one embodiment of the invention, a mimetic may be identified by a method comprising the steps of: a) contacting sclerostin with a candidate mimetic; and b) measuring a signaling response induced by the sclerostin-mimetic interaction, wherein a signaling response that is at least 10% (e.g., equal to or more than 20%, 30%, 40%, 50%, 60%) of the signaling response measured for the sclerostin:sclerostin-binding-partner interaction indicates the candidate mimetic is identified as a sclerostin-binding-partner mimetic of the invention.

The signaling response is preferably the response of the Wnt and/or BMP pathway, in which case a mimetic would cause a decrease in Wnt and/or BMP pathway activities compared with the non-stimulated state. The signaling response can be determined, for example, by measuring signaling levels using a reporter construct as already mentioned above. When sclerostin binds a sclerostin-binding-partner mimetic, inhibiting the Wnt and/or BMP pathway, expression of a reporter protein is inhibited, which reduction can be measured, for example, by immunoassay, fluorescence, light measurement, etc., depending on the nature of the reporter protein. The expression can also be measured for the sclerostin:sclerostin-binding-partner interaction.

Any of the binding assays described can be used to determine the presence of a mimetic in a sample, e.g., a tissue sample, that binds to sclerostin. To do so, sclerostin is reacted in the presence or absence of the sample, and signaling is measured as appropriate for the assay being used. An increase of 10% or more (e.g., equal to or more than 20%, 30%, 40%, 50%, 60%) in the signaling of sclerostin indicates that the sample contains a mimetic that binds to sclerostin.

Any of the signaling assays described can also be used to determine the presence of a mimetic in a library of compounds. Such screening techniques using, for example, high throughput screening are well known in the art.

The present invention provides additionally a method for diagnosing a disorder or predisposition to a sclerostin-related disorder and/or to an aberrant bone mineral density disorder in a subject comprising the steps of: (a) obtaining the nucleotide sequence of a sclerostin-binding-partner gene in said subject, and (b) comparing it to that of a healthy subject, where a mutation in the respective sclerostin-binding-partner gene indicates a sclerostin-related disorder or a predisposition thereto.

Mutations in SOST or gene encoding a sclerostin-binding-partner in a subject may be predictors of developing a disorder relating to abnormal bone mass, and/or can be used to make a diagnosis. Such mutations change the interactions between the sclerostin and sclerostin-binding-partner, e.g., cause an increase or decrease in binding and signaling compared with a healthy subject.

One embodiment of the present invention is a method for diagnosing a disorder or susceptibility to a disorder relating to abnormal bone mass in a subject comprising the step of obtaining the DNA nucleotide sequence of SOST or gene encoding a sclerostin-binding-partner in said subject and comparing it to that of a healthy subject, where a mutation in the respective sclerostin or gene encoding a sclerostin-binding-partner indicates a disorder relating to abnormal bone mass or a susceptibility thereto.

Another embodiment of the present invention is a method for diagnosing a disorder or susceptibility to a disorder relating to abnormal bone mass in a subject comprising the step of obtaining the DNA nucleotide sequence of SOST or gene encoding a sclerostin-binding-partner in said subject and comparing it to that of a healthy subject, where a presence of a mutation that changes binding respectively to sclerostin or a sclerostin-binding-partner compared with a healthy subject indicates a disorder relating to abnormal bone mass or a susceptibility thereto.

Mutations may be present in the non-translated portions of a gene (e.g., in the introns, control sequences, promoters) as these also lead to a dysfunction in the expressed protein. Such mutations may be single nuclear polymorphisms (SNPs).

The mutation may have the effect of decreasing interaction between sclerostin and sclerostin-binding-partner. Compared with a healthy subject, the binding may be at least 10%, 20%, 30%, 40%, 50%, 60%, 70%, 80%, and preferably at least 20% lower than the binding observed in a healthy subject. Where a decrease in binding is observed, a disorder relating to low bone mass can be diagnosed or predicted in the case of a sclerostin-binding partner that increases sclerostin action. Conversely, in the case of a sclerostin-binding partner that decreases sclerostin action, when a decrease in binding is observed, a disorder relating to high bone mass can be diagnosed or predicted.

The mutation may have the effect of increasing the signaling response of the Wnt and/or BMP pathways. Compared with a healthy subject, the signaling response may be at least 10%, 20%, 30%, 40%, 50%, 60%, 70%, 80%, and preferably at least 20% higher than the response observed in a healthy subject. Where an increase in response is observed, a disorder relating to high bone mass can be diagnosed or predicted.

Alternatively, the mutation may have the effect of increasing the binding between a sclerostin-binding-partner and sclerostin. Compared with a healthy subject, the binding may be at least 10%, 20%, 30%, 40%, 50%, 60%, 70%, 80%, and preferably at least 20% higher than the binding observed in a healthy subject. Where an increase in binding is observed, a disorder relating to low bone mass can be diagnosed or predicted in the case of a sclerostin-binding partner that increases sclerostin action. Conversely, in the case of a sclerostin-binding partner that decreases sclerostin action, when a decrease in binding is observed, a disorder relating to high bone mass can be diagnosed or predicted.

The mutation may have the effect of decreasing the signaling response of the Wnt and/or BMP pathways. Compared with a healthy subject, the signaling response may be at least 10%, 20%, 30%, 40%, 50%, 60%, 70%, 80%, and preferably at least 20% lower than the response observed in a healthy subject. Where a decrease in response is observed, a disorder relating to low bone mass can be diagnosed or predicted.

Binding and signaling assays are within the routine practices of the skilled person, and are described above. Methods of sequencing specific genes is well known and described, for example, in Sambrook et al., Molecular Cloning: A Laboratory Manual, Cold Spring Harbor Laboratory Press, New York (1989).

Candidate Agents and Compounds

The candidate or test compounds or agents of or employed by the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al. (1997) Anticancer Drug Des. 12: 145).

Examples of methods for the synthesis of molecular libraries can be found in the art, for example in: DeWitt et al. (1993) Proc. Natl. Acad. Sci. U.S.A. 90: 6909; Erb et al. (1994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and in Gallop et al. (1994) J. Med. Chem. 37: 1233.

Libraries of compounds may be presented in solution (e.g., Houghten (1992) Biotechniques 13: 412), or on beads (Lam (1991) Nature 354: 82), chips (Fodor (1993) Nature 364: 555), bacteria (Ladner, U.S. Pat. No. 5,223,409), spores (Ladner '409), plasmids (Cull et al. (1992) Proc Natl Acad Sci USA 89: 1865) or on phage (Scott and Smith (1990) Science 249: 386); (Devlin (1990) Science 249: 404); (Cwirla et al. (1990) Proc. Natl. Acad. Sci. 87: 6378); (Felici (1991) J. Mol. Biol. 222: 301); (Ladner, supra).

In one embodiment, an assay is a cell-based assay comprising contacting a cell expressing a sclerostin-binding-partner with a candidate or test compound or agent, and determining the ability of the test compound to modulate (e.g. stimulate or inhibit) the activity of said sclerostin-binding-partner. Determining the ability of the test compound to modulate the sclerostin-binding-partner can be accomplished, for example, by determining the ability of the candidate or test compound or agent to modulate the sclerostin:sclerostin-binding-partner interaction.

Determining the ability of candidate or test compounds or agents to modulate a sclerostin-binding-partner can be accomplished by determining direct binding. These determinations can be accomplished, for example, by coupling the sclerostin-binding-partner protein with a radioisotope or enzymatic label such that binding of the protein to a candidate or test compound or agent can be determined by detecting the labeled protein in a complex. For example, molecules, e.g., proteins, can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

It is also within the scope of this invention to determine the ability of candidate or test compounds or agents to modulate a sclerostin-binding-partner (or the sclerostin:sclerostin-binding-partner interaction), without the labeling of any of the interactants. For example, a microphysiometer can be used to detect the interaction of test compounds with a sclerostin-binding-partner without the labeling of any of the interactants (McConnell et al. (1992) Science 257: 1906). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In yet another embodiment, an assay of the present invention is a cell-free assay in which a protein or biologically active portion thereof is contacted with a candidate or test compound or agent (e.g., or a compound tested for its ability to modulate a sclerostin-binding-partner, or to modulate signaling resulting from the sclerostin:sclerostin-binding-partner interaction) and the ability of the test compound to bind to the sclerostin-binding-partner, or biologically active portions thereof, is determined. Binding of the test compound to the sclerostin-binding-partner proteins can be determined either directly or indirectly as described above.

Such a determination may be accomplished using a technology such as real-time Biomolecular Interaction Analysis (BIA). Sjolander et al., 1991 Anal. Chem. 63:2338-2345 and Szabo et al., 1995 Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize sclerostin or a sclerostin-binding-partner to facilitate separation of complexed from uncomplexed forms of the protein, as well as to accommodate automation of the assay. Binding of a test compound to sclerostin or a sclerostin-binding-partner can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, glutathione-S-transferase/kinase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and the non-adsorbed sclerostin or a sclerostin-binding-partner protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding determined using standard techniques.

Other techniques for immobilizing proteins on matrices can also be used in the screening assays of the invention. For example, sclerostin or a sclerostin-binding-partner can be immobilized utilizing conjugation of biotin and streptavidin. Biotinylated sclerostin or sclerostin-binding-partner protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with sclerostin or sclerostin-binding-partner proteins or target molecules can be derivatized to the wells of the plate, and unbound sclerostin or sclerostin-binding-partner protein trapped in the wells by antibody conjugation. Methods for detecting such complexes, in addition to those described above for the GST-immobilized complexes, include immunodetection of complexes using antibodies reactive with the sclerostin or sclerostin-binding-partner protein or target molecules.

In yet another aspect of the invention, the sclerostin or sclerostin-binding-partner proteins can be used as “bait proteins” in a two-hybrid assay or three-hybrid assay (see, e.g., U.S. Pat. No. 5,283,317; Zervos et al., 1993 Cell 72:223-232; Madura et al., 1993 J. Biol. Chem. 268:12046-12054; Bartel et al., 1993 Biotechniques 14:920-924; Iwabuchi et al., 1993 Oncogene 8:1693-1696; and Brent WO94/10300), to identify other proteins which bind to sclerostin or a sclerostin-binding-partner. Such sclerostin or sclerostin-binding-partner-binding proteins are also likely to be involved in the propagation of signals by the sclerostin or sclerostin-binding-partner proteins.

The two-hybrid system is based on the modular nature of most transcription factors, which consist of separable DNA-binding and activation domains. Briefly, the assay utilizes two different DNA constructs. In one construct, the gene that codes for a sclerostin or sclerostin-binding-partner protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a kinase dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the sclerostin or sclerostin-binding-partner protein which interacts with the protein.

This invention further pertains to novel agents identified by the above-described screening assays. Accordingly, it is within the scope of this invention to further use an agent identified as described herein in an appropriate animal model. For example, an agent identified as described herein (e.g., an agent capable of modulating the sclerostin:sclerostin-binding-partner interaction) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Pharmaceutical Compositions

A composition as described herein may be a pharmaceutical composition. The invention provides for pharmaceutical compositions comprising (i) a modulator (e.g., a small molecule modulator) of the sclerostin:sclerostin-binding-partner interaction, or (ii) a sclerostin-binding-partner mimetic according to the invention admixed with a physiologically compatible carrier. In addition to the active ingredients, these pharmaceutical compositions may contain a significant amount of one or more inorganic or organic, solid or liquid, pharmaceutically acceptable carriers, and physiologically acceptable diluents (such as water, phosphate buffered saline, or saline), which can be used pharmaceutically.

The pharmaceutical compositions according to the invention are suitable for administration to a warm-blooded mammal, especially a human (or to cells or cell lines derived from a warm-blooded mammal, especially a human, e.g. osteoblasts or osteoclasts), for the treatment, amelioration, diagnosis, or prevention of a sclerostin-related disorder and/or an aberrant bone mineral density disorder.

The pharmaceutical compositions according to the invention are those for enteral, such as nasal, rectal or oral, or parenteral, such as intramuscular or intravenous, administration to warm-blooded mammals (especially a human). The dose of the active ingredient depends on the species of warm-blooded mammal, the body weight, the age and the individual condition, individual pharmacokinetic data, the disease to be treated and the mode of administration. For instance, the dose of a modulator (e.g., small molecule modulator) or a pharmaceutically acceptable salt thereof to be administered to warm-blooded mammals, for example humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

Appropriate dosages can also be determined in trials, first in an appropriate animal model, and subsequently in the species to be treated. The amount and frequency of administration will depend, of course, on such factors as the nature and severity of the indication being treated, the desired response, the condition of the individual being treated, and so forth. The appropriate dosages are within the range of about 10 ng/kg/day to about 100 μg/kg/day each or in combination. Preferably a dose of 100 ng/kg/day to about 1000 ng/kg/day for 1-20 days can be expected to induce an appropriate biological effect. Alternatively, bolus injections of from about 1 μg/kg/day to about 100 μg/kg/day can be given at approximately 4-day intervals to exert antimicrobial effects via augmentation of immune and/or inflammatory responses mediated by macrophages/monocytes.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragèes, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefosse, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragèe cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragèe cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragèe coatings or the capsule casings, for example for identification purposes or to indicate different doses of active ingredient.

Antibodies

Sclerostin-binding-partners can be used as immunogens to generate antibodies using standard techniques for polyclonal and monoclonal antibody preparation. The full length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of the amino acid sequence of the sclerostin-binding-partner, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. Hydropathy plots or similar analyses can be used to identify hydrophilic regions.

An immunogen typically is used to prepare antibodies by immunizing a suitable subject, (e.g., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

As used herein, the term antibody refers to immunoglobulin molecules and immunologically active portions of immunoglobulin molecules, i.e., molecules that contain an antigen binding site which specifically binds an antigen, such as a sclerostin-binding-partner. Antibody includes conventional immunoglobulin molecule, as well as fragments thereof which are also specifically reactive with sclerostin-binding-partner and/or sclerostin. Antibodies can be fragmented using conventional techniques and the fragments screened for utility in the same manner as described herein below for whole antibodies. Examples of immunologically active portions of immunoglobulin molecules include F(ab) and F(ab′)2 fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well known techniques, such as protein A chromatography to obtain the IgG fraction. At an appropriate time after immunization, e.g., when the specific antibody titers are highest, antibody producing cells can be obtained from the subject and used to prepare monoclonal antibodies by standard techniques, such as the hybridoma technique originally described by Kohler and Milstein (1975) Nature 256:495 497, the human B-cell hybridoma technique (Kozbor et al. (1983) Immunol. Today 4:72), the EBV hybridoma technique (Cole et al. (1985), Monoclonal Antibodies and Cancer Therapy, Alan R. Liss, Inc., pp. 77 96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27 9400 01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370 1372; Hay et al. (1992) Hum. Antibod. Hybridomas 3:81 85; Huse et al. (1989) Science 246:1275 1281; Griffiths et al. (1993) EMBO J. 12:725 734.

Additionally, recombinant antibodies, such as chimeric and humanized monoclonal antibodies, comprising both human and non human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041 1043; Liu et al. (1987) Proc. Natl. Acad. Sci. USA 84:3439 3443; Liu et al. (1987) J. Immunol. 139:3521 3526; Sun et al. (1987) Proc. Natl. Acad. Sci. USA 84:214 218; Nishimura et al. (1987) Canc. Res. 47:999 1005; Wood et al. (1985) Nature 314:446 449; and Shaw et al. (1988) J. Natl. Cancer Inst. 80:1553 1559); Morrison (1985) Science 229:1202 1207; Oi et al. (1986) Bio/Techniques 4:214; U.S. Pat. No. 5,225,539; Jones et al. (1986) Nature 321:552 525; Verhoeyan et al. (1988) Science 239:1534; and Beidler et al. (1988) J. Immunol. 141:4053 4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899 903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody-Like (e.g., Non-Immunoglobulin) Scaffolds

A wide variety of antibody/immunoglobulin frameworks or scaffolds can be employed so long as the resulting polypeptide includes at least one binding region which is specific for the target protein. Such frameworks or scaffolds include the 5 main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the invention pertains to generating non-immunoglobulin based scaffold molecules by screening non-immunoglobulin scaffolds libraries against a sclerostin-binding partner. For screening non-immunoglobulin scaffolds libraries, similar display technologies used for antibody libraries, including but not limited to phage display, ribosome display, RNA display or yeast display, can be used. In another aspect, the invention pertains to generating non-immunoglobulin based antibodies using non-immunoglobulin scaffolds onto which CDRs of the invention can be grafted. Known or future non-immunoglobulin frameworks and scaffolds may be employed, as long as they comprise a binding region specific for the target protein. Such compounds are known herein as “polypeptides comprising a target-specific binding region.” or Antibody-like Scaffold. Known non-immunoglobulin frameworks or scaffolds include, but are not limited to, fibronectin III-based derived molecules such as Adnectins (fibronectin) (Adnexus, Inc., Waltham, Mass.), ankyrin (Molecular Partners AG, Zurich, Switzerland), domain antibodies (Domantis, Ltd (Cambridge, Mass.) and Ablynx nv (Zwijnaarde, Belgium)), lipocalin (Anticalin) (Pieris Proteolab AG, Freising, Germany), small modular immuno-pharmaceuticals (Trubion Pharmaceuticals Inc., Seattle, Wash.), maxybodies (Avidia, Inc. (Mountain View, Calif.)), Protein A (Affibody AG, Sweden) and affilin (gamma-crystallin or ubiquitin) (Scil Proteins GmbH, Halle, Germany).

According to the instant invention, the anti-sclerostin or anti-sclerostin-binding-partner protein antibody or fragment thereof, or the polypeptide comprising a sclerostin-specific or a sclerostin-binding-partner-specific binding region, regardless of the framework or scaffold employed, may be bound, either covalently or non-covalently, to an additional moiety. The additional moiety may be a polypeptide, an inert polymer such as PEG, small molecule, radioisotope, metal, ion, nucleic acid or other type of biologically relevant molecule. Such a construct, which may be known as an immunoconjugate, immunotoxin, or the like, is also included in the meaning of antibody, antibody fragment or polypeptide comprising a sclerostin-specific or a sclerostin-binding-partner-specific binding region, as used herein.

(i) Fibronectin Type III-Based Scaffold

The fibronectin type III-based scaffolds are based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands. (U.S. Pat. No. 6,818,418).

These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the invention using standard cloning techniques.

(ii) Ankyrin—Molecular Partners

The technology is based on using proteins with ankyrin derived repeat modules as scaffolds for bearing variable regions which can be used for binding to different targets. The ankyrin repeat module is a 33 amino acid polypeptide consisting of two anti-parallel alpha-helices and a turn. Binding of the variable regions is mostly optimized by using ribosome display.

(iii) Maxybodies/Avimers—Avidia

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, 20040175756; 20050053973; 20050048512; and 20060008844.

(vi) Protein A—Affibody

Affibody® affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate Affibody® libraries with a large number of ligand variants (See e.g., U.S. Pat. No. 5,831,012). Affibody® molecules mimic antibodies, they have a molecular weight of 6 kDa, compared to the molecular weight of antibodies, which is 150 kDa. In spite of its small size, the binding site of Affibody® molecules is similar to that of an antibody.

(v) Anticalins—Pieris

Anticalins® are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids.

The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain.

The set of four loops, which makes up the binding pocket, shows pronounced structural plasticity and tolerates a variety of side chains. The binding site can thus be reshaped in a proprietary process in order to recognize prescribed target molecules of different shape with high affinity and specificity.

One protein of lipocalin family, the bilin-binding protein (BBP) of Pieris Brassicae has been used to develop anticalins by mutagenizing the set of four loops. One example of a patent application describing “anticalins” is PCT WO 199916873.

(vi) Affilin—Scil Proteins

Affilin™ molecules are small non-immunoglobulin proteins which are designed for specific affinities towards proteins and small molecules. New Affilin™ molecules can be very quickly selected from two libraries, each of which is based on a different human derived scaffold protein.

Affilin™ molecules do not show any structural homology to immunoglobulin proteins. Scil Proteins employs two Affilin™ scaffolds, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Fusion Proteins

The invention provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in frame to each other. The heterologous polypeptide can be fused to the N terminus or C terminus of the polypeptide of the invention.

One useful fusion protein is a GST fusion protein in which the polypeptide of the invention is fused to the C terminus of GST sequences. Such fusion proteins can facilitate the purification of a recombinant polypeptide of the invention.

In another embodiment, the fusion protein contains a heterologous signal sequence at its N terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction may be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in frame to the polypeptide of the invention.

An example of such fusion proteins is a protein fusion comprising the extracellular part of LRP4, e.g., the extracellular part consisting of SEQ ID NO:3. An example of such fusion protein is the polypeptide of SEQ ID NO:4.

RNAi

The invention provides small interfering ribonucleic acid sequences (siRNA), as well as compositions and methods for inhibiting the expression of the SOST gene or genes encoding sclerostin-binding-partners in a cell or mammal using the siRNA. The invention also provides compositions and methods for treating pathological conditions and diseases in a mammal caused by the aberrant expression of the SOST gene or genes encoding sclerostin-binding-partners, or caused by the aberrant signaling of pathways of which said genes are integral members, using siRNA. siRNA directs the sequence-specific degradation of mRNA through a process known as RNA interference (RNAi).

The siRNA of the invention comprises an RNA strand (the antisense strand) having a region which is less than 30 nucleotides in length, generally 19-24 nucleotides in length, and is substantially complementary to at least part of an mRNA transcript of the SOST gene or genes encoding sclerostin-binding-partners. The use of these siRNAs enables the targeted degradation of mRNAs of genes that are implicated in the sclerostin, BMP, or Wnt signaling pathways.

The siRNA molecules according to the present invention mediate RNA interference (“RNAi”). The term “RNAi” is well known in the art and is commonly understood to mean the inhibition of one or more target genes in a cell by siRNA with a region which is complementary to the target gene. Various assays are known in the art to test siRNA for its ability to mediate RNAi (see for instance Elbashir et al., Methods 26 (2002), 199-213). The effect of the siRNA according to the present invention on gene expression will typically result in expression of the target gene being inhibited by at least 10%, 33%, 50%, 90%, 95% or 99% when compared to a cell not treated with the RNA molecules according to the present invention.

“siRNA” or “small-interfering ribonucleic acid” according to the invention has the meanings known in the art, including the following aspects. The siRNA consists of two strands of ribonucleotides which hybridize along a complementary region under physiological conditions. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

The siRNA molecules in accordance with the present invention comprise a double-stranded region which is substantially identical to a region of the mRNA of the target gene. A region with 100% identity to the corresponding sequence of the target gene is suitable. This state is referred to as “fully complementary.” However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Another factor affecting the efficiency of the RNAi reagent is the target region of the target gene. The region of a target gene effective for inhibition by the RNAi reagent may be determined by experimentation. A suitable mRNA target region would be the coding region. Also suitable are untranslated regions, such as the 5′-UTR, the 3′-UTR, and splice junctions. For instance, transfection assays as described in Elbashir S. M. et al, 2001 EMBO J., 20, 6877-6888 may be performed for this purpose. A number of other suitable assays and methods exist in the art which are well known to the skilled person.

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention confer a high in vivo stability suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918 and will not be repeated here. Suitable modifications for oral delivery are more specifically set out in the Examples and description herein. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates). Finally, end modifications sometimes referred to herein as 3′ caps or 5′ caps may be of significance. Caps may consist of more complex chemistries which are known to those skilled in the art.

In one embodiment, the invention provides double-stranded ribonucleic acid (dsRNA) molecules for inhibiting the expression of the SOST gene or genes encoding sclerostin-binding-partners. The dsRNA comprises at least two sequences that are complementary to each other. The dsRNA comprises a sense strand comprising a first sequence and an antisense strand comprising a second sequence. The antisense strand comprises a nucleotide sequence which is substantially complementary to at least part of an mRNA encoding SOST gene or genes encoding sclerostin-binding-partners, and the region of complementarity is less than 30 nucleotides in length, generally 19-24 nucleotides in length. The dsRNA, upon contacting with a cell expressing the SOST gene or genes encoding sclerostin-binding-partners, inhibits the expression of said genes by at least 40%.

In another embodiment, the invention provides a cell comprising one of the dsRNAs of the invention. The cell is generally a mammalian cell, such as a cell from a mouse, rat, rabbit, sheep, cow or primate.

In another embodiment, the invention provides a pharmaceutical composition for inhibiting the expression of the SOST gene or genes encoding sclerostin-binding-partners in an organism, generally a human subject, comprising one or more of the dsRNA of the invention and a pharmaceutically acceptable carrier or delivery vehicle.

In another embodiment, the invention provides a method for inhibiting the expression of the SOST gene or genes encoding sclerostin-binding-partners in a cell, comprising the following steps:

(a) introducing into the cell a double-stranded ribonucleic acid (dsRNA), wherein the dsRNA comprises at least two sequences that are complementary to each other. The dsRNA comprises a sense strand comprising a first sequence and an antisense strand comprising a second sequence. The antisense strand comprises a region of complementarity which is substantially complementary to at least a part of a mRNA encoding sclerostin or sclerostin-binding-partners, and wherein the region of complementarity is less than 30 nucleotides in length, generally 19-24 nucleotides in length, and wherein the dsRNA, upon contact with a cell expressing the SOST gene or genes encoding sclerostin-binding-partners, inhibits expression of said genes by at least 40%; and

(b) maintaining the cell produced in step (a) for a time sufficient to obtain degradation of the mRNA transcript of the SOST gene or genes encoding sclerostin-binding-partners, thereby inhibiting expression of said genes in the cell.

In another embodiment, the invention provides methods for treating, preventing or managing pathological processes mediated by sclerostin, BMP, or Wnt signaling, e.g. sclerostin-related disorders and/or aberrant bone mineral density disorders, comprising administering to a patient in need of such treatment, prevention or management a therapeutically or prophylactically effective amount of one or more of the siRNAs of the invention.

In another embodiment, the invention provides vectors for inhibiting the expression of the SOST gene or genes encoding sclerostin-binding-partners in a cell, comprising a regulatory sequence operably linked to a nucleotide sequence that encodes at least one strand of one of the siRNA of the invention.

Inhibitory nucleic acid compounds of the present invention may be synthesized by conventional means on a commercially available automated DNA synthesizer, e.g. an Applied Biosystems (Foster City, Calif.) model 380B, 392 or 394 DNA/RNA synthesizer, or like instrument. Phosphoramidite chemistry may be employed. The inhibitory nucleic acid compounds of the present invention may also be modified, for instance, nuclease resistant backbones such as e.g., phosphorothioate, phosphorodithioate, phosphoramidate, or the like, described in many references may be used. The length of the inhibitory nucleic acid has to be sufficient to ensure that the biological activity is inhibited. Thus, for instance in case of antisense oligonucleotides, has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Double-stranded RNA, i.e., sense-antisense RNA, also termed small interfering RNA (siRNA) molecules, can also be used to inhibit the expression of nucleic acids for SOST gene or genes encoding sclerostin-binding-partners. RNA interference is a method in which exogenous, short RNA duplexes are administered where one strand corresponds to the coding region of the target mRNA (Elbashir et al. (2001) Nature 411: 494). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism. Preferred siRNA molecules are typically from 19 to 25 nucleotides long, preferably about 21 nucleotides in length. Effective strategies for delivering siRNA to target cells include, for example, transduction using physical or chemical transfection.

Alternatively siRNAs may be expressed in cells using, e.g., various PolIII promoter expression cassettes that allow transcription of functional siRNA or precursors thereof. See, for example, Scherr et al. (2003) Curr. Med. Chem. 10(3):245; Turki et al. (2002) Hum. Gene Ther. 13(18):2197; Cornell et al. (2003) Nat. Struct. Biol. 10(2):91. The invention also covers other small RNAs capable of mediating RNA interference (RNAi) such as for instance micro-RNA (miRNA) and short hairpin RNA (shRNA).

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The following examples are merely illustrative and not meant to limit the scope of the present claims in any manner.

### EXAMPLES

### Example 1

### Discovery of Association Between Sclerostin and Sclerostin-Binding-Partners

In order to identify novel modulators of the sclerostin, BMP, and Wnt pathways, we applied a systematic tandem affinity purification (TAP) method to sclerostin. As described in Rigaut et al. (Nat. Biotechnol. (1999) 17(10): 1030), Seraphin and Rigaut WO 00/09716 Patent application) the contents of which are hereby incorporated by reference, the TAP purification method involves the fusion of the TAP tag to the target protein of interest and the introduction of the construct into the cognate host cell or organism.

The TAP tag is a tandem fusion of (i) IgG-binding units of protein A from Staphylococcus aureus (ProtA); and (ii) the Calmodulin Binding Peptide (CBP), separated by a TEV protease cleavage site. It allows the rapid purification of complexes from a relatively small number of cells without prior knowledge of the complex composition, activity, or function. Combined with mass spectrometry, the TAP strategy allows for the identification of proteins interacting with a given target protein.

Both N and C terminally tagged sclerostin were expressed in HEK293T and in osteoblastic UMR-106 cells non-treated or treated with 50 ng/ml BMP-2 for 4 and 20 hours. The N-terminal TAP-tag in addition contains an artificial signaling sequence from the CD33 protein. UMR-106 and HEK293T cells were chosen, since we found previously that UMR-106 and related HEK293 cells express endogenous SOST mRNA. (Keller, H. and Kneissel, M (2005) Bone 37:148).

The proper sub-cellular localization of both SOST TAP-tagged proteins was monitored by indirect immunofluorescence. Pre-tests demonstrated that sufficient amounts of TAP-tagged sclerostin were found in the biochemical prepared membrane fraction to start a TAP approach. Furthermore, both tagging constructs of sclerostin were found to be secreted in the media of either cell type. The expression level of C-TAP sclerostin in the membrane fraction from UMR-106 cells was found to be insufficient for a TAP purification. Further processing of those samples was discontinued.

Cells were grown in DMEM medium with 10% FCS. For stimulation the medium was replaced with either fresh medium containing 50 ng/ml BMP2 or fresh medium alone. Cells were stimulated for 4 hours and 20 hours before harvesting by mechanical detachment, washed with excess PBS on ice and lysed in immunoprecipitation buffer.

The raw lysate underwent a subcellular fractionation to enrich for membrane associated proteins.

The membrane fraction was used to perform the TAP purification. For purification of secreted TAP-tagged SOST complexes the cell culture supernatant was collected from several cell culture plates.

Triplicate purifications were performed for the membrane fraction, while cell culture supernatant purifications were performed in unicates only.

Purified protein complexes were separated by 1D-SDS-PAGE and stained by colloidal coomassie blue. Entire gel lanes were systematically cut into slices and proteins were digested in-gel with trypsin as described in Shevchenko (Shevchenko, A., Wilm, M., Vorm, O. & Mann, M. Mass spectrometric sequencing of proteins silver-stained polyacrylamide gels. Anal. Chem. 68, 850-858 (1996)). Protein identification was performed by LC-MS/MS, and MS data were searched against an in-house curated version of the International Protein Index (IPI), maintained at the EBI (Hinxton, UK). Results of database searches were read into a database system for further bioinformatics analysis (including subjecting the hits to Computer Aided Target Selection (CATS) analysis).

About 100 proteins are identified in total from both cell lines with an E-value≦10, R-value≧0.67.

Hits were culled into a short list after being filtered against ribosomal proteins and RNA binding proteins, and other abundant cytoplasmic proteins. Sclerostin seemed to show a propensity for RNA binding proteins (about 60 candidates are left with an E-value≦10). The values used herein are as follows:

IPI=Protein reference number from the IPI database

R=Reproducibility of the identification within the set of triplicate purifications

E=Total number of entry points the protein was identified by Tandem Affinity Purifications for a given reference dataset.

MS=Number of peptides the respective proteins was identified by MS

TABLE I
Short List of Membrane Purification

| | | | | | | | | | | | | | | | | | UMR- | | UMR- | |
|---|---|---|---|---|---|---|---|---|---|---|---|---|---|---|---|---|---|---|---|---|
| | | | | | 293T | | 293T | | | | 293T | | 293T | | | | 106 | | 106 | |
| | | | | | CD33 | | CD33 | | | | C-TAP | | C-TAP | | UMR- | | CD33 | | CD33 | |
| | | | 293T | | BMP2 | | BMP2 | | 293T | | BMP2 | | BMP2 | | 106 | | BMP2 | | BMP2 | |
| | | | CD33 | | (4 h) | | (20 h) | | C-TAP | | (4 h) | | (20 h) | | CD33 | | (4 h) | | (20 h) | |
| | | E- | R-value/ | | R-value/ | | R-value/ | | R-value/ | | R-value/ | | R-value/ | | R-value/ | | R-value/ | | R-value/ | |
| IPI | Name | value | MS | | MS | | MS | | MS | | MS | | MS | | MS | | MS | | MS | |
| IPI00414021.1 | TRIM41 | 1 | 1 | 7 | 0.33 | 3 | 1 | 3 | | | 0.33 | 2 | | | | | 0.33 | 3 | | |
| (Human) | | | | | | | | | | | | | | | | | | | | |
| IPI00019439.1 | FBN2 | 1 | | | | | | | 0.33 | 1 | 1 | 2 | | | | | | | | |
| (Human) | | | | | | | | | | | | | | | | | | | | |
| IPI00006288.1 | SLIT2 | 1 | | | | | | | | | 0.67 | 3 | 0.33 | 1 | | | | | | |
| (Human) | | | | | | | | | | | | | | | | | | | | |
| IPI00304993.3 | IL17R | 1 | | | | | | | 0.67 | 2 | 0.33 | 1 | 0.67 | 1 | | | | | | |
| (Human) | | | | | | | | | | | | | | | | | | | | |
| IPI00010948.2 | TRIM26 | 2 | 1 | 22 | 1 | 8 | 0.67 | 11 | 1 | 8 | 1 | 8 | 1 | 4 | | | | | | |
| (Human) | | | | | | | | | | | | | | | | | | | | |
| IPI00180707.7 | FREM2 | 2 | | | | | | | 1 | 4 | 0.67 | 2 | 0.67 | 2 | | | | | | |
| (Human) | | | | | | | | | | | | | | | | | | | | |
| IPI00306851.3 | LRP4 | 2 | 0.33 | 2 | | | | | 0.33 | 1 | 0.33 | 1 | | | 0.67 | 4 | 0.67 | 2 | | |
| (Human) | | | | | | | | | | | | | | | | | | | | |
| IPI00153032.1 | C6orf93 | 3 | 0.67 | 10 | 1 | 4 | 1 | 12 | 0.33 | 3 | 0.33 | 2 | 1 | 5 | | | 0.67 | 5 | 0.33 | 2 |
| (Human) | | | | | | | | | | | | | | | | | | | | |
| IPI00000203.1 | LRP6 | 3 | | | | | | | 0.67 | 4 | 1 | 4 | | | 0.33 | 2 | 0.33 | 1 | 0.33 | 1 |
| (Human) | | | | | | | | | | | | | | | | | | | | |
| IPI00024292.1 | LRP2 | 5 | | | 0.67 | 2 | 0.33 | 2 | | | | | | | | | | | | |
| (Human) | | | | | | | | | | | | | | | | | | | | |

TABLE II
Short List of Supernatant Purification

| | | | | | 293T | | | | | | | | | | | | | |
|---|---|---|---|---|---|---|---|---|---|---|---|---|---|---|---|---|---|---|
| | | | | | CD33 | | | | | | | | UMR-106 | | | | UMR-106 | |
| | | | 293T | | BMP2 | | 293T | | | | UMR-106 | | CD33 | | UMR-106 | | CTAP | |
| | | | CD33 | | (20 h) | | CTAP | | | | CD33 | | BMP2 (20 h) | | CTAP | | BMP2 (20 h) | |
| | | E- | R-value/ | | R-value/ | | R-value/ | | 293T CTAP | | R-value/ | | R-value/ | | R-value/ | | R-value/ | |
| IPI | Name | value | MS | | MS | | MS | | BMP2 (20 h) | | MS | | MS | | MS | | MS | |
| IPI00137336.1 (Mouse) | Gpc1 | 1 | | | | | | | | | 1 | 19 | | | 1 | 9 | 1 | 13 |
| IPI00009802.1 (Human) | CSPG2 | 1 | 1 | 33 | 1 | 23 | 1 | 19 | 1 | 6 | | | | | | | | |
| IPI00374563.2 (Human) | AGRN | 1 | 1 | 5 | | | | | 1 | 16 | | | | | | | | |
| IPI00199629.1 (Rat) | Sdc4 | 1 | | | | | | | | | 1 | 2 | | | | | | |
| IPI00203479.3 (Rat) | Serpine2 | 1 | | | | | | | | | | | 1 | 1 | 1 | 3 | | |
| IPI00403938.1 (Mouse) | Tnc | 5 | | | | | | | | | 1 | 3 | 1 | 4 | 1 | 4 | 1 | 4 |
| IPI00024292.1 (Human) | LRP2 | 5 | 1 | 9 | | | | | | | | | | | | | | |
| IPI00327143.1 (Rat) | Alpl | 1 | | | | | | | | | 1 | 11 | 1 | 2 | 1 | 2 | 1 | 3 |

As seen in Table I and Table II, the following sclerostin interaction partners were identified by this approach: Versican (CSPG2), FREM2, Fibrillin 2 (FBN2), C6orf93, Syndecan-4 (Sdc4), Agrin (AGRN), Serpine-2 (PN-1), LRP2, LRP4, LRP6, SLIT2, tenascin C, TRIM26, TRIM41, glypican1, alkaline phosphatase (ALPL) and IL-17 receptor.

Either, the effect of interaction partner down-regulation (siRNA) or up-regulation (overexpression) on the action of sclerostin in the Wnt1/STF assay have been tested in HEK293 (human embryonic kidney cells), C28a2 (human chondrocytes cell line where no sclerostin could be detected) and/or UMR106 (rat osteosarcoma cells) cells, either a biochemical assay has been performed (example: ALPL)

### Example 2

### LRP4 Data

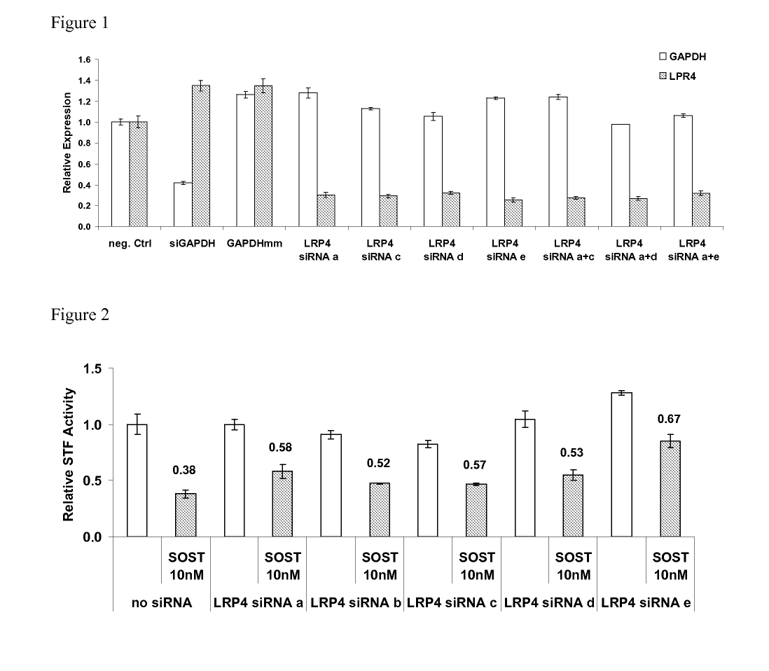

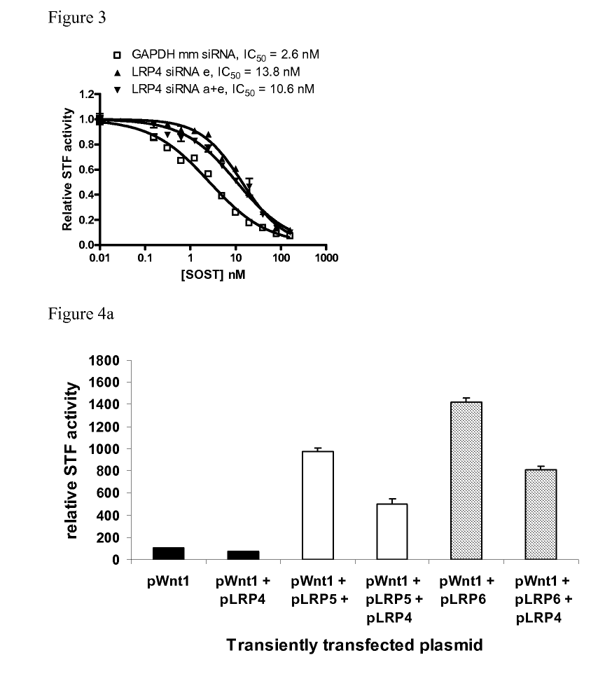

Briefly, siRNAs were screened against LRP4 in a Wnt1 induced Wnt signaling reporter assay (supertopflash (STF)) in HEK293 cells. All siRNAs against LRP4 were able to knockdown LRP4 mRNA (FIG. 1). LRP4 mRNA knockdown reduced the ability of sclerostin to inhibit STF activity in HEK293 cells (FIG. 2). A sclerostin dose response study showed that, as compared to the control, LRP4 knockdown resulted in up to 5-fold increase in IC₅₀ of SOST in the STF/Wnt1 assay (FIG. 3).

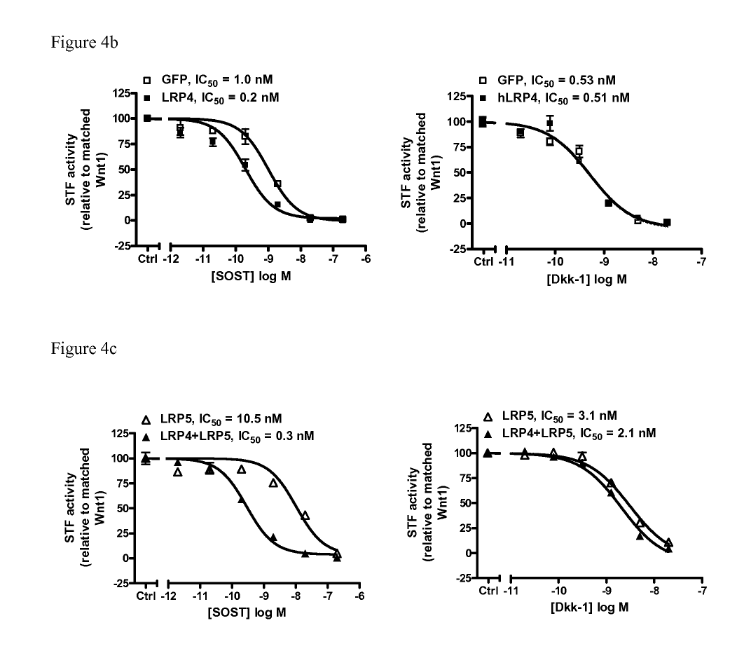

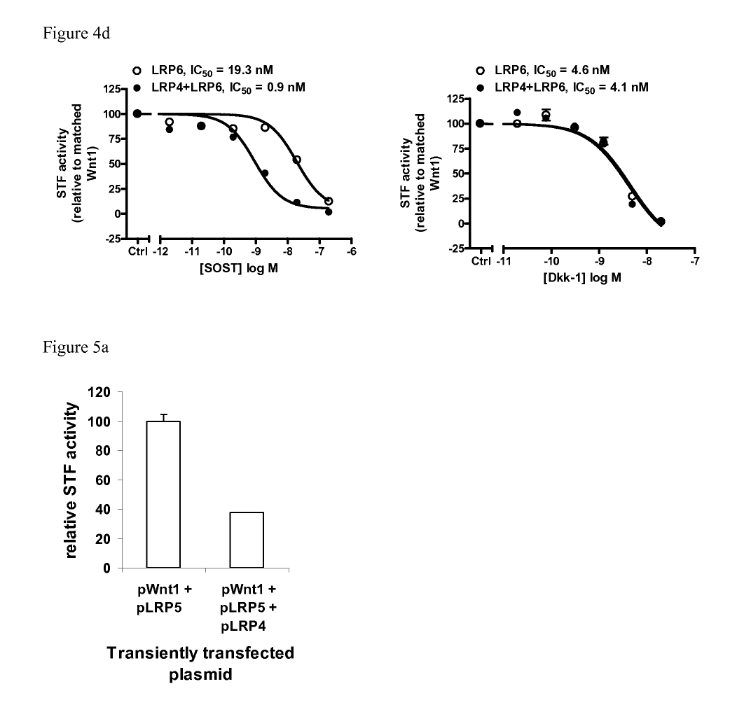

LRP4 overexpression in HEK293 cells decreased Wnt signaling as measured by STF assay (FIG. 4a). Overexpression of LRP4 in HEK293 cells resulted in a 5-fold decrease in SOST IC₅₀ and was without effect on Dkk1 IC₅₀ (FIG. 4b). Overexpression of LRP4 together with LRP5 in HEK cells was without effect on Dkk1 IC₅₀ but resulted in a 35-fold decrease in SOST IC₅₀ compared to control cells overexpressing only LRP5 (FIG. 4c). Overexpression of LRP4 together with LRP6 in HEK cells was without effect on Dkk1 IC₅₀ but resulted in a 20-fold decrease in SOST IC₅₀ compared to control cells overexpressing only LRP6 (FIG. 4d). These data demonstrate that LRP4 is a specific facilitator of sclerostin action in HEK cells.

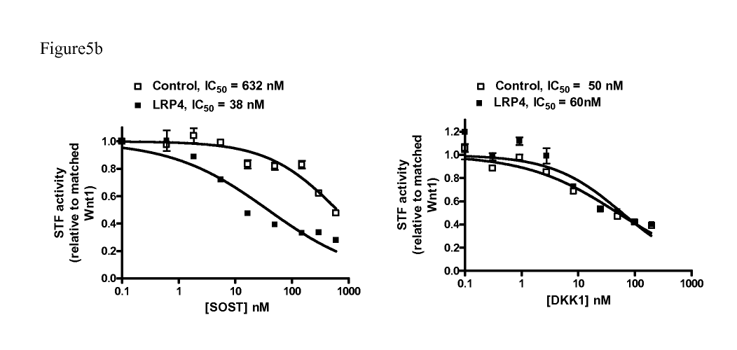

LRP4 overexpression in SOST-free C28a2 cells induced a 2.5-fold decrease in canonical Wnt signaling as measured by STF assay in cells transiently transfected with LRP5 (FIG. 5a). Overexpression of LRP4 and LRP5 in C28a2 cells was without effect on Dkk1 IC₅₀ but resulted in a 16-fold decrease in SOST IC₅₀ compared to control cells overexpressing only LRP5 (FIG. 5b). The efficacy of SOST action at 600 nM was increased from 52% to 72%. These data demonstrate therefore that LRP4 is a facilitator of sclerostin action in C28a2 cells, while it does not affect DKK1 activity.

Based on these findings, LRP4 is hypothesized to be an important interaction partner for sclerostin, enhancing sclerostin action. Consequently, modulation of this interaction may provide novel ways to inhibit sclerostin action in skeletal tissues.

### Example 3

### Alkaline Phosphatase Data

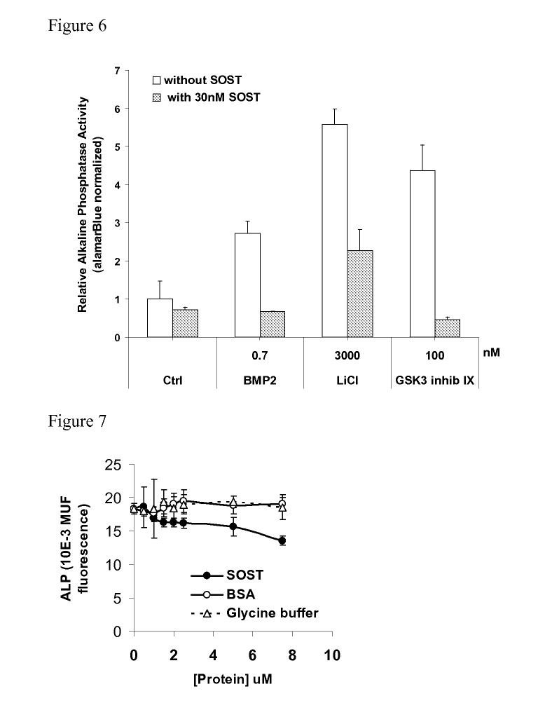

The effect of sclerostin on alkaline phosphatase was in addition tested in a cell-based alkaline phosphatase assay in MC3T3 cells. This assay is based on the detection of the activity of the endogenous alkaline phosphatase by measuring spectrophotometrically the dephosphorylation of p-nitrophenyl phosphate. To test whether sclerostin could inhibit alkaline phosphatase downstream of BMP, Wnt and LRP5/6, the effect of sclerostin on GK3beta inhibitor-induced alkaline phosphatase were tested (FIG. 6). Sclerostin decreased LiCl-induced alkaline phosphatase and GSK-3 Inhibitor IX (Calbiochem #361550)-induced alkaline phosphatase. We tested then the effect of sclerostin on ALPL itself in a cell-free assay, based on the fluorescent detection of the dephosphorylation of 4-methylumbelliferyl phosphate (FIG. 7). A sclerostin dose response study showed that, as compared to control, high concentrations of sclerostin inhibit alkaline phosphatase activity. These data suggest a direct inhibitory effect of SOST on alkaline phosphatase activity.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

We claim:

1. A method of treating a pathological disorder that is mediated by sclerostin or that is associated with an abnormal level of sclerostin in a patient, the method comprising the step of administering to the patient a composition comprising a modulator that modulates binding of LRP4 to sclerostin.

2. The method of claim 1, wherein the pathological disorder is an aberrant bone mineral density disorder, osteoporosis, sclerosteosis, cancer, myeloma, or multiple myeloma with osteolytic lesions.

3. The method of claim 1, wherein the pathological disorder is characterized by increased activity or overexpression of sclerostin, and wherein the modulator decreases expression of LRP4.

4. The method of claim 1, wherein the pathological disorder is characterized by increased activity or overexpression of sclerostin, and wherein the modulator decreases binding of LRP4 to sclerostin.

5. The method of claim 1, wherein the pathological disorder is characterized by decreased activity or decreased expression of sclerostin, and wherein the modulator increases expression of LRP4.

6. The method of claim 1, wherein the pathological disorder is characterized by decreased activity or decreased expression of sclerostin, and wherein the modulator increases binding of LRP4 to sclerostin.

7. The method of claim 1, wherein the modulator is an antibody, an antibody-like scaffold, small molecule, chimeric or fusion protein, peptide, mimetic, or inhibitory nucleotide.

8. The method of claim 1, wherein the modulator is an antibody or a functional fragment thereof that binds to LRP4.

9. The method of claim 1, the modulator is a monoclonal antibody or a functional fragment thereof that binds to LRP4.

10. The method of claim 1, the modulator is a recombinant, chimeric, humanized, or human antibody or a functional fragment thereof that binds to LRP4.

11. The method of claim 1, wherein the modulator is an agent that modulates the Wnt signaling pathway as measured in a cell-based assay.

12. The method of claim 1, wherein the modulator decreases expression of LRP4.

13. The method of claim 3, wherein the modulator is a siRNA to LRP4.

14. The method of claim 1, wherein the modulator is a soluble fragment of LRP4 that binds to sclerostin and inhibits binding of LRP4 to sclerostin.

15. The method of claim 1, wherein modulator is a soluble fragment of LRP4 that consists of the extracellular portion of LRP4.

16. The method of claim 15, wherein the sequence of the fragment consists of the sequence of SEQ ID NO: 3.

17. The method of claim 1, wherein the composition further comprises a pharmaceutically acceptable carrier.


SEQUENCE LISTING (4 sequences)

SEQ ID NO: 1 - protein, 1950 aa, Homo sapiens
MRRQWGALLLGALLCAHAVALGLRAGERTRSGPGSSSPSGGISGGASAGSGLGRGAGLGRGAGLASSPECACGRSHFTCAVSALGECTCIPAQWQCDGDNDCGDHSDEDGCILPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEENCPSAVPAPPCNLEEFQCAYGRCILDIYHCDGDDDCGDWSDESDCSSHQPCRSGEFMCDSGLCINAGWRCDGDADCDDQSDERNCTTSMCTAEQFRCHSGRCVRLSWRCDGEDDCADNSDEENCENTGSPQCALDQFLCWNGRCIGQRKLCNGVNDCGDNSDESPQQNCRPRTGEENCNVNNGGCAQKCQMVRGAVQCTCHTGYRLTEDGHTCQDVNECAEEGYCSQGCTNSEGAFQCWCETGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQNLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKISSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASGRQVIISSNLTWPNGLAIDYGSQRLYWADAGMKTIEFAGLDGSKRKVLIGSQLPHPFGLTLYGERIYWTDWQTKSIQSADRLTGLDRETLQENLENLMDIHVFHRRRPPVSTPCAMENGGCSHLCLRSPNPSGFSCTCPTGINLLSDGKTCSPGMNSFLIFARRIDIRMVSLDIPYFADVVVPINITMKNTIAIGVDPQEGKVYWSDSTLHRISRANLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRAVLINNNLGWPNGLTVDKASSQLLWADAHTERIEAADLNGANRHTLVSPVQHPYGLTLLDSYIYWTDWQTRSIHRADKGTGSNVILVRSNLPGLMDMQAVDRAQPLGFNKCGSRNGGCSHLCLPRPSGFSCACPTGIQLKGDGKTCDPSPETYLLFSSRGSIRRISLDTSDHTDVHVPVPELNNVISLDYDSVDGKVYYTDVFLDVIRRADLNGSNMETVIGRGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVSHVSHPFALTQQDRWIYWTDWQTKSIQRVDKYSGRNKETVLANVEGLMDIIVVSPQRQTGTNACGVNNGGCTHLCFARASDFVCACPDEPDSRPCSLVPGLVPPAPRATGMSEKSPVLPNTPPTTLYSSTTRTRTSLEEVEGRCSERDARLGLCARSNDAVPAAPGEGLHISYAIGGLLSILLILVVIAALMLYRHKKSKFTDPGMGNLTYSNPSYRTSTQEVKIEAIPKPAMYNQLCYKKEGGPDHNYTKEKIKIVEGICLLSGDDAEWDDLKQLRSSRGGLLRDHVCMKTDTVSIQASSGSLDDTETEQLLQEEQSECSSVHTAATPERRGSLPDTGWKHERKLSSESQV

SEQ ID NO: 2 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 3 - protein, 1744 aa, Homo sapiens
ALGLRAGERTRSGPGSSSPSGGISGGASAGSGLGRGAGLGRGAGLASSPECACGRSHFTCAVSALGECTCIPAQWQCDGDNDCGDHSDEDGCILPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEENCPSAVPAPPCNLEEFQCAYGRCILDIYHCDGDDDCGDWSDESDCSSHQPCRSGEFMCDSGLCINAGWRCDGDADCDDQSDERNCTTSMCTAEQFRCHSGRCVRLSWRCDGEDDCADNSDEENCENTGSPQCALDQFLCWNGRCIGQRKLCNGVNDCGDNSDESPQQNCRPRTGEENCNVNNGGCAQKCQMVRGAVQCTCHTGYRLTEDGHTCQDVNECAEEGYCSQGCTNSEGAFQCWCETGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQNLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKISSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASGRQVIISSNLTWPNGLAIDYGSQRLYWADAGMKTIEFAGLDGSKRKVLIGSQLPHPFGLTLYGERIYWTDWQTKSIQSADRLTGLDRETLQENLENLMDIHVFHRRRPPVSTPCAMENGGCSHLCLRSPNPSGFSCTCPTGINLLSDGKTCSPGMNSFLIFARRIDIRMVSLDIPYFADVVVPINITMKNTIAVGVDPQEGKVYWSDSTLHRISRANLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRAVLINNNLGWPNGLTVDKASSQLLWADAHTERIEAADLNGANRHTLVSPVQHPYGLTLLDSYIYWTDWQTRSIHRADKGTGSNVILVRSNLPGLMDMQAVDRAQPLGFNKCGSRNGGCSHLCLPRPSGFSCACPTGIQLKGDGKTCDPSPETYLLFSSRGSIRRISLDTSDHTDVHVPVPELNNVISLDYDSVDGKVYYTDVFLDVIRRADLNGSNMETVIGRGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVGHVSHPFALTQQDRWIYWTDWQTKSIQRVDKYSGRNKETVLANVEGLMDIIVVSPQRQTGTNACGVNNGGCTHLCFARASDFVCACPDEPDSQPCSLVPGLVPPAPRATGMSEKSPVLPNTPPTTLYSSTTRTRTSLEEVEGRCSERDARLGLCARSNDAVPAAP

SEQ ID NO: 4 - protein, 1998 aa, Homo sapiens
MPLLLLLPLLWAGALAALGLRAGERTRSGPGSSSPSGGISGGASAGSGLGRGAGLGRGAGLASSPECACGRSHFTCAVSALGECTCIPAQWQCDGDNDCGDHSDEDGCILPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEENCPSAVPAPPCNLEEFQCAYGRCILDIYHCDGDDDCGDWSDESDCSSHQPCRSGEFMCDSGLCINAGWRCDGDADCDDQSDERNCTTSMCTAEQFRCHSGRCVRLSWRCDGEDDCADNSDEENCENTGSPQCALDQFLCWNGRCIGQRKLCNGVNDCGDNSDESPQQNCRPRTGEENCNVNNGGCAQKCQMVRGAVQCTCHTGYRLTEDGHTCQDVNECAEEGYCSQGCTNSEGAFQCWCETGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQNLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKISSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASGRQVIISSNLTWPNGLAIDYGSQRLYWADAGMKTIEFAGLDGSKRKVLIGSQLPHPFGLTLYGERIYWTDWQTKSIQSADRLTGLDRETLQENLENLMDIHVFHRRRPPVSTPCAMENGGCSHLCLRSPNPSGFSCTCPTGINLLSDGKTCSPGMNSFLIFARRIDIRMVSLDIPYFADVVVPINITMKNTIAVGVDPQEGKVYWSDSTLHRISRANLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRAVLINNNLGWPNGLTVDKASSQLLWADAHTERIEAADLNGANRHTLVSPVQHPYGLTLLDSYIYWTDWQTRSIHRADKGTGSNVILVRSNLPGLMDMQAVDRAQPLGFNKCGSRNGGCSHLCLPRPSGFSCACPTGIQLKGDGKTCDPSPETYLLFSSRGSIRRISLDTSDHTDVHVPVPELNNVISLDYDSVDGKVYYTDVFLDVIRRADLNGSNMETVIGRGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVGHVSHPFALTQQDRWIYWTDWQTKSIQRVDKYSGRNKETVLANVEGLMDIIVVSPQRQTGTNACGVNNGGCTHLCFARASDFVCACPDEPDSQPCSLVPGLVPPAPRATGMSEKSPVLPNTPPTTLYSSTTRTRTSLEEVEGRCSERDARLGLCARSNDAVPAAPLEVLFQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK